SEQUENCE LISTING (15 sequences)

SEQ ID NO: 1 - DNA, 224 bp, Sus scrofa
gaattgaccaggtcttgtggagaaaacagatccagacggcaaacatacgcaagggatttagtcaaacacatttttggcaaaaaaactatgaattttgtaatcagttgtgagccaatgaaatacaaaaatgagtctagttaataatctacaattattggttaaagaagtatattagtgctgactttcctctgttcgtcctaccttttcttttctatcaaccccac

SEQ ID NO: 2 - DNA, 2241 bp, Artificial
  Chimeric transgene construct comprising porcine albumin promoter/enhancer operably linked to mutant Herpes virus thymidine kinase (xTK) gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactgcaggaattgaccaggtcttgtggagaaaacagatccagacggcaaacatacgcaagggatttagtcaaacacatttttggcaaaaaaactatgaattttgtaatcagttgtgagccaatgaaatacaaaaatgagtctagttaataatctacaattattggttaaagaagtatattagtgctgactttcctctgttcgtcctaccttttcttttctatcaaccccacatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaactgcccacgctactgcgggtttatatagacggtctccacgggctggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagaattcgctagctctctagtcgagaattcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctaagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggcctt

SEQ ID NO: 3 - DNA, 1506 bp, Artificial
  Chimeric transgene construct comprising porcine alpha-fetoprotein promoter/enhancer operably linked to fungal cytosine deaminase gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactgcagtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacaccatggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtaggctagctctctagtcgagaattcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaat

SEQ ID NO: 4 - DNA, 2294 bp, Artificial
  Chimeric transgene construct comprising cytomegalovirus promoter operably linked to mutant Herpes virus thymidine kinase (xTK) gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagataatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggcctt

SEQ ID NO: 5 - DNA, 1782 bp, Artificial
  Chimeric transgene construct comprising cytomegalovirus promoter operably linked to fungal cytosine deaminase gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatcctgcagatggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtaggctagctctctagtcgagtccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattcttattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 1269 bp, Sus scrofa
  CDS (16)..(1128)
catgaggagaaaataatgaatgtcaaaggaagagtggttctgtcaatgctgcttgtctcaactgtaatggttgtgttttgggaatacatcaacagcccagaaggttctttgttctggatataccagtcaaaaaacccagaagttggcagcagtgctcagaggggctggtggtttccgagctggtttaacaatgggactcacagttaccacgaagaagaagacgctataggcaacgaaaaggaacaaagaaaagaagacaacagaggagagcttccgctagtggactggtttaatcctgagaaacgcccagaggtcgtgaccataaccagatggaaggctccagtggtatgggaaggcacttacaacagagccgtcttagataattattatgccaaacagaaaattaccgtgggcttgacggtttttgctgtcggaagatacattgagcattacttggaggagttcttaatatctgcaaatacatacttcatggttggccacaaagtcatcttttacatcatggtggatgatatctccaggatgcctttgatagagctgggtcctctgcgttcctttaaagtgtttgagatcaagtccgagaagaggtggcaagacatcagcatgatgcgcatgaagaccatcggggagcacatcctggcccacatccagcacgaggtggacttcctcttctgcatggacgtggatcaggtcttccaaaacaactttggggtggagaccctgggccagtcggtggctcagctacaggcctggtggtacaaggcacatcctgacgagttcacctacgagaggcggaaggagtccgcagcctacattccgtttggccagggggatttttattaccacgcagccatttttgggggaacacccactcaggttctaaacatcactcaggagtgcttcaagggaatcctccaggacaaggaaaatgacatagaagccgagtggcatgatgaaagccatctaaacaagtatttccttctcaacaaacccactaaaatcttatccccagaatactgctgggattatcatataggcatgtctgtggatattaggattgtcaagatagcttggcagaaaaaagagtataatttggttagaaataacatctgactttaaattgtgccagcagttttctgaatttgaaagagtattactctggctacttcctcagagaagtagcacttaattttaacttttaaaaaaatactaacaaaataccaacacagtaagtacatattattcttcctt

SEQ ID NO: 7 - DNA, 1121 bp, Ovis aries
  CDS (11)..(1120)
ggagaaaataatgaatgtcaaaggaaaagtgattctgtcaatgctggttgtctcaactgtcattgttgtgttttgggaatatatccacagcccagaaggctctttgttctggataaacccatcaagaaacccagaagtcagtggcggcagcagcattcagaagggctggtggtttccgagatggtttaacaatggttaccaagaagaagatgaagacgtagacgaagaaaaggaacaaagaaaggaagacaaaagcaagcttaagctatcggactggttcaacccatttaaacgccctgaggttgtgactatgacagattggaaggcacccgtggtgtgggaaggcacttacaacagagccgtcttagacgattactacgccaagcagaaaattaccgtcggcctgacggttttcgccgtcggaagatacattgagcattacttggaggagttcttaacgtctgctaataagcacttcatggttggccaccgagtcatcttttacgtcatggtggacgatgtctccaggatgcctttgatagagctgggccctctgcgctccttcaaagtgtttgaggtcaagcctgagaggaggtggcaggacgtcagcatggtgcgcatgaagaccatcggggagcacatcgtggcccacatccaacgtgaggttgacttcctcttctgcatggacgtggaccaggtcttccaagatgagttcggggtggagaccctgggtgagtcggtggcccagctacaggcctggtggtacaaggcagatcccgatgagtttacctacgagaggcgcaaggagtctgcagcatatattcccttcggcgaaggggatttttattaccacgcagccatttttgggggaacacccactcaggtccttaacatcacccaggaatgcttcaaaggaatcctcaaggacaagaaaaatgacatagaagcccaatggcatgatgagagccatctaaacaagtatttccttctcaacaaacccactaaaatcttatccccggaatactgctgggattatcatataggcctacctgcggatattaagcttgtcaagatgtcttggcagacaaaagagtataatttggttagaaataatgtctgac

SEQ ID NO: 8 - DNA, 1655 bp, Bos taurus
  CDS (469)..(1575)
ccgggggccgggccgagctgggagcgtcgagcccgctgcccagcgcccgccggctccctcgcgcccctgcccgccgccccggaggagcgcccggcggccggccgacgggagcgcagcggcacaccccgccccggcacgcccgcggggctcgggaggaggcagcgcgccgactgttccggcagccgaggacgccgccggggagccgaggcgccggccagcccccagcgcgcccagcttctgcggatcagggaaaccacgtgtcctcaagtggccagccagctgtccccaagaggaacttgcctggcatttgcacggaaagacgagacacttcacaaaatcaacggagtcagaaggctgcaccttcgcttcctcccagccctgcctccttctgcagaacggagctcagtagaacttggtacttttgccttttactctaggaggagagaagcagacgatgaggagaaaataatgaatgtcaaaggaaaagtgattctgtcaatgctggttgtctcaactgtcattgttgtgttttgggaatatatccacagcccagaaggctctttgttctggataaacccatcaagaaacccagaagttggtggcagcagcattcagaagggctggtggcttccgagatggtttaacaatggttaccatgaagaagatggagacataaacgaagaaaaggaacaaagaaacgaagacgaaagcaagcttaagctatcggactggttcaacccatttaaacgccccgaggttgtgaccatgacgaagtggaaggctccagtggtgtgggaaggcacttacaacagagccgtcttagacaattattatgccaagcagaaaattaccgtcggcctgacggttttcgccgtcggaagatacattgagcattacttggaggagttcttaacgtctgctaataagcacttcatggtgggccacccagtcatcttttatatcatggtagatgatgtctccaggatgcctttgatagagttgggtcctctgcgctccttcaaagtgtttaagatcaagcctgagaagaggtggcaggacatcagcatgatgcgcatgaagactatcggggagcacattgtggcccacatccagcatgaggttgacttccttttctgcatggatgtggaccaggtcttccaagacaagtttggggtggagaccctgggcgagtcggtggcccagctacaagcctggtggtacaaggcagatcccaatgacttcacctacgagaggcggaaggagtctgcagcatacattcccttcggcgaaggggatttttattaccatgcagccatttttgggggaacacccactcaggtccttaacatcacccaggaatgcttcaaaggaatcctcaaggacaagaaaaatgacatagaagcccaatggcatgatgaaagccatctaaacaagtatttccttctcaacaaacctactaaaatcttatccccggaatactgctgggattatcacataggcctacctgcggatattaagcttgtcaagatgtcttggcagacaaaagagtataatgtggttagaaataatgtctgactttgtgccagtacatttctgaatttgagagagtattattctggctacttcctcagaaaagtaacacttaattttaactt

SEQ ID NO: 9 - DNA, 3450 bp, Mus musculus
  CDS (445)..(1560)
cgtcttaggaggctggagattctgggtggagccctagccctgccttttcttagctggctgacaccttcccttgtagactcttcttggaatgagaagtaccgattctgctgaagacctcgcgctctcaggctctgggagttggaaccctgtaccttcctttcctctgctgagccctgcctccttcggcaggccagagctcgacagaagctcggttgctttgctgtttgctttggagggaacacagctgacgatgaggctgactttgaactcaagagatctgcttaccccagtctcctggaattaaaggcctgtactaccttgcctggacctaagattttcatgatcactatgcttcaagatctccatgtcaacaagatctccatgtcaagatccaagtcagaaacaagtcttccatcctcaagatctggatcacaggagaaaataatgaatgtcaagggaaaagtaatcctgttgatgctgattgtctcaaccgtggttgtcgtgttttgggaatatgtcaacagcccagacggctctttcttgtggatatatcacacaaaaattccagaggttggtgagaacagatggcagaaggactggtggttcccaagctggtttaaaaatgggacccacagttatcaagaagacaacgtagaaggacggagagaaaagggtagaaatggagatcgcattgaagagcctcagctatgggactggttcaatccaaagaaccgcccggatgttttgacagtgaccccgtggaaggcgccgattgtgtgggaaggcacttatgacacagctctgctggaaaagtactacgccacacagaaactcactgtggggctgacagtgtttgctgtgggaaagtacattgagcattacttagaagactttctggagtctgctgacatgtacttcatggttggccatcgggtcatattttacgtcatgatagatgacacctcccggatgcctgtcgtgcacctgaaccctctacattccttacaagtctttgagatcaggtctgagaagaggtggcaggatatcagcatgatgcgcatgaagaccattggggagcacatcctggcccacatccagcacgaggtcgacttcctcttctgcatggacgtggatcaagtctttcaagacaacttcggggtggaaactctgggccagctggtagcacagctccaggcctggtggtacaaggccagtcccgagaagttcacctatgagaggcgggaactgtcggccgcgtacattccattcggagagggggatttttactaccacgcggccatttttggaggaacgcctactcacattctcaacctcaccagggagtgctttaaggggatcctccaggacaagaaacatgacatagaagcccagtggcatgatgagagccacctcaacaaatacttccttttcaacaaacccactaaaatcctatctccagagtattgctgggactatcagataggcctgccttcagatattaaaagtgtcaaggtagcttggcagacaaaagagtataatttggttagaaataatgtctgacttcaaattgtgatggaaacttgacactattactctggctaattcctcaaacaagtagcaacacttgatttcaacttttaaaagaaacaatcaaaaccaaaacccactaccatggcaaacagatgatttctcctgacaccttgagcctgtaatatgtgagaaagagtctatggcaagtaatcaggtataaattctcaatgatttcttatatattctgggtcttgggaaaacttgattctagaaatcaaaattaatttgacaaaggaaaagcagatgccggaaacttcttcccagtctgtcatacaattcaccactggccaggtgctgagagaagcattagggaacagtgtgggttgtgtcagagttggacggctccatccctttggcttcattatcttcctcctcatggagattctaaagcaacccagagaggctttgcagccagagacctttaataaggatgccaatgtgaccatcagtctgtaaaagctgatggctccaggagcgctggcagtccaggccccactaggctattgtttctgtcctgggcataaaggaggcagagagtgccaataggtactttggtggcacatgttcagagtccaggaaaaatcaagggtgaccacttagagggacataggacttggggttggtgattgaactgagttacaaacacagacagctttcttcaggatgactaacagcaggaattgaatggaaagtgtgttcattttgttttgcccaaattgtattcatgctgttagctttgtgtgttgagccctgtggagagggtgtgactgtatcagggaaggagagtacctcagcggactgaggaccagcaccctattatatcagaagacaatctctcatcatcaggtcctacctacaacctgctctgaacctccgagttcctcagcccatcgtgttccagtgtgggggcctgtatggagcaggtgactgaagacaaagccccctgtcacatgacctcatttcccctgctctagtactatgcaagtgtgacagccagccagccagatgtactggacaacataggaaccgactttatggcaatgggagccgcagtcactacaacggagctgctgaaggttctgttccccgctctgagagcctgcaggagcccctgtataggtggttctcaacctatgggtcgcgacccctttgggaagtgttaaatgaccctttcacaggtgtcccctaagacggttaaaaaacatagatatttccactctgactggtaacagtagcagaattacagttatgaaatagcaagggaaataattctggggttcgtgtcatccataccatgaggagctacattaggtcacatcattagggaagttgagaagcatagctctacttgggtatttaagcaaattatgcaaagggggttgtcgctctgtgttctgtgtatgcatatatttatattttgcttgtcttccagtttaggtcaatctgtttcttcctttaagcagtttatttaaaaggccattgcaccatcttggtgaacagcatgaggggtttcaataaaaaataggatcttacctttgtccacagggctctacctcttacttttcaattgtgaacaaaaaaggtcgcacacccagaggcaacaaaacccacagaattcctgaaccaatgggagatgccaatggaagcagagcttgcacatctgctaaaaattctgcctctctgtcactgtgctggatccgtctaaagtgggacagttcaatggtctgaaagtttcaaaaaggctggggaatttgaggggatttttttttaaaataaaattgatccaagtttaaatctctaatgagtaagcttaggattttattaaaggtaatttttagacattcttcaaaataagaattc

SEQ ID NO: 10 - DNA, 1936 bp, Rattus norvegicus
  CDS (78)..(1097)
tttcgagcaggtcatttcttctatctctctgggctggaaaaaactgcatggcaggttttcaccactgacttctccccatggctctggagggactcagggccaagaagagactcctgtggcgactcttcctgtctgcgtttggtctcctaggcctgtaccattactggttcaagattttcaggctctttgaagtgttcatccccatgggcatctgccctatggccatcatgcctctgctaaaggacaatttcaccggagtgctgcgtcactgggcccggcctgaagtcctgacctgtacctcttggggagccccaattatctgggatgagaccttcgacccacacgtagctgagcgagaggcgagacggcagaaccttaccattggactgactgtctttgcggtaggcaggtacctggagaagtacctggagcacttcctggtatcggcagagcagtacttcatggtcggccagaacgtggtgtattatgtgtttaccgaccgaccggaagcagtgccccacgtggctctaggccagggtcgcctgctgcgggtgaaaccagtgagaagagagaagcgctggcaggacgtgtcaatggctcgcatgctcacgctacacgaggctctgggagggcagctgggccgagaagctgactatgtgttctgcctggacgtggaccagtacttcagcggtaacttcggacccgaggtgctggcagatttggtggcacagctgcacgcctggcacttccgctggcctcggtggatgctgccctacgagagggacaagcggtcggctgccgcgctgtcgttaagcgaaggcgatttctactaccacgccgcggtgttcgggggcagtgtggcggctctgctcaagctgacggctcactgtgcgactggccaacagctggaccgtgagcatggcattgaggcacgctggcacgacgagagccacctcaacaagttcttctggctgagcaagcccaccaagctgctgtcgccagagttttgctgggcagaggaaattggctggaggccagagatccaccaccctcgtctgatctgggcacccaaggagtacgcactggtgcgaacctagcaggccaccctggccaggcgggcacaggaacgtctttggcactgcgagtgttgaatggaattgcaaaatgtttcaaaagcacgaagtcagagccagttacactgcctttggaagggggacttcggaactcagaggtactgtgcctggggccgtacacccttgtcagctggccagagagcctttttgctccaaccagatgcaaattatggaaaggcaagagtttgtcagcttactttgcctacaaggccttccttagcccttggtaatggttggtgtctcagcttctgtccggaccttaggtttgacagctgtctggagccattcctgtgaccaggacctgcagattctcttctcttgctcttcacggtcttctgttctacttcagcccccagctctccattctctgcctgtgctaacatgcagctccctgtctgcatttgttcaactgcttcttcctccgtcatcaggaaggtttccattcctgcagctgggctggcttcctgacccaatgagcacttccccttcagctcccaagcagctggctgccttgctgctgttgtagtgtggctggcagaccactcggttgtcacggcctctgccaccaccacggctgtctcttcatgttcctagacagccctcagccttggcaaggccattcaagggaagcctcttgtgggacccttcagctcggtcggtgtcatgtaggagcacgggagagtgttggctctcctccccaaccccatccctgcatgctagctcactaagggcagctgatcatctagttttacagccataaaagaggtgtttaagccaaaaaaaaaaaaaaaaaaaaaaaaaaa

SEQ ID NO: 11 - DNA, 1113 bp, Mus musculus
  CDS (1)..(1113)
atggctctggggacagagttgggagtgagctggccagggtcacatggaagttgccgagaacaagaaggacagagacaaagaggcccagggaagccaacctggggactttcacgggccaagaagagactcctgtggcggttcttcctgtctgcatttggtttcttaggcctgtaccattacaggttcattattatcaggctcatagaaggctccatccccatgggcacctgccctacagccataatgcctctgccgagggacaatttcacaggagtactgcaccactgggcccggcctgaagtcctgacctgtacctcttggggagcaccaattatttgggatggcactttcgaccctcatgtagcccagcaagaggcgagacggcggaacctcaccatcgggctgactgtgtttgctgtaggcaggtacctggagaagtacctggaacacttcctggtatcggcagagcagcacttcatggtcggccagaacgtggtgtactatgtgtttacggatcgcccggaagcagtgccctatgtggctctaggccagggtcgcctgctgcgggcaaaacccgtgcagcgagagaggcgctggcaggacgtgtccatggcacgcatgcccacgctacacgaggctctgggagggcagctgggccaagaagctgactttgtgttctgcctggacgtggaccagtacttcaccggtaacttcgggcctgaggtgctggcagatttggtggcacagctgcacgcctggcactaccgctggccgcggtggctgctgccctacgagagggacaagcgatcggctgctgcgctgtcgttaagcgaaggcgatttctactaccacgctgcggtgtttggcggcagtgtggctgcactgctcaaactgacggcccactgtgcgactggccaacagctggaccataagcgcggcattgaggcactctggcacgacgaaagccaccttaacaagttcttctggctgaacaagcccaccaagctgctgtcgcctgagttctgctgggcagaggaaattatctggaggagagagatccatcacccacgcctgctctgggcacccaaggaatatacgctggtgcgaaactag

SEQ ID NO: 12 - DNA, 672 bp, Sus sp.
  CDS (1)..(672)
ctagttgtagaaaaggatgaagaaaatggagttttgcttctagaactaaatcctcctaacccgtgggattcagaacccagatctcctgaagatttggcttttggggaagtgcagatcacgtaccttactcacgcctgcatggacctcaagctgggagacaagaggatggtgttcgatccttggttaatcggtcctgcttttgcgcgaggatggtggttactacacgagcctccatctgattggctggagaggctgagccgcgcagatttaatttacatcagtcacatgcactcagaccacctgagttacccaacactgaagaagcttgctgagagaagaccagatgttcccatttatgttggcaacacggaaagacctgtattttggaatctgaatcagagtggcgtccagttgactaatatcaatgtagtgccatttggaatatggcagcaggtagacaaaaatcttcgattcatgatcttgatggatggcgttcatcctgagatggacacttgcattattgtggaatacaaaggtcataaaatactcaatacagtggattgcaccagacccaatggaggaaggctgcctatgaaggttgcattaatgatgagtgattttgctggaggagcttcaggctttccaatgactttcagtggtggaaaatttact

SEQ ID NO: 13 - DNA, 2282 bp, Mus musculus
  CDS (461)..(2191)
ggaccctccattctggcagtggcagctgtgaagtctagcgatcttcttctggagagaacagatcagcagggtggagctgggagaggggtagtgttgatgttagacacataatgtgacatcagcctgctctgcatctgtgtctccagaggaacttgaacttcccagctgtactttggtaattgcttctgaaaaaaacctcctggccacacagaaatcaaataaaatttttaatcaagcaaattgaaaaatccaggctgtattataaacatcaattaaaagaagatctgggcctgggccttttgatgtttcctgaccagcttggatgctgtcacctttgtctactcagaagggagtaaaggcagtctaaagtcatcctcgccatcctcgccttcctggtgtgattcgcagaagtagaaagaacaccagcagctgctttgaaataccctggagctggcagatgatggacaggaaacagacagctgagaccctgctgaccctgtctcctgctgaagttgccaacctcaaggaagggatcaatttttttcgaaataagactactgggaaagagtacattttatacaaggagaaggaccatctaaaggcatgcaagaacctctgcaagcaccagggaggcctgttcatgaaagacatcgaggatttagatggaaggtccgttaaatgcacaaagcacaactggaagttagacgtgagcaccatgaaatatatcaaccctccagggagcttctgtcaagacgagctcgttattgaaatggatgaaaacaatgggctttccctggtagaactgaaccctcctaacccctgggactctgatcccaggtctcctgaagaattagcttttggggaagtacagataacatatctcactcatgcctgcatggacctcaagttgggagacaagcgaatggtatttgacccttggttaattggccctgcttttgcccgaggatggtggttgctacatgagcctccatctgactggttggagaggctgtgcaaagcagacctcatttatatcagccacatgcactcagaccacctgagctaccctaccctgaagcagctttcccagagacgaccagacattcccatttatgttggcgacacagaaaggcctgtgttttggaacctggatcagagtggcgtcgggttaactaacatcaacgtggttccatttggaatatggcaacaggtagacaaaagtctgcggttcatgatcttgatggacggcgttcatcctgagatggacacatgcattatcgtggagtacaaaggtcataaaatactcaacacagtggactgcaccagacccaatgggggaaggcttcctgagaaagttgctctaatgatgagtgatttcgcaggaggtgcatcaggctttccaatgactttcagtggtggaaaatttactgaggaatggaaagcccagttcattaaggctgaaagaagaaagcttctgaattacaaagctcagctggtgaaggacctgcagccccgaatctactgtccgtttgctgggtactttgtggagtctcacccatctgacaagtacattaaggaaacaaacaccaaaaatgacccaaatcagctcaacaatcttatcaggaaaaactctgacgtggtgacatggaccccacgacctggcgctgtcctcgaccttggcaggatgctgaaggacccaacagacagcaagggcattgtggagcctccagaggggacaaagatttacaaggattcctgggactttggcccgtacctggagatcttgaattctgctgtcagagatgaaatcttctgtcattcatcctggattaaagagtacttcacgtgggctggatttaagaattacaacctggtggtcaggatgattgaaacagatgaagatttcagcccttttcctggagggtacgactatctggtggactttctagatttatcctttccgaaagaaagacccagccgggagcatccttatgaagaaatccatagccgggtggatgtcatcaggtacgtggtgaagaacggcctgctgtgggatgatctgtatattggattccagacccgattgctgcgggaccctgatatataccatcatctgttttggaatcattttcagataaaactccctctaacaccacccaactggaagtcgttcctaatgcactgtgattagtctggacctggtaagtcccaggaccccagcccagaggatggtgcctgaacattcaagatgggtctcccctgcttcgataaacctttctgga

SEQ ID NO: 14 - DNA, 2444 bp, Mus musculus
  CDS (623)..(2353)
ggaccctccattctggcagtggcagctgtgaagtctagcgatcttcttctggagagaacagatcagcaggcaataatggagtgttcccggtcaactctcatggcctcaggactctcacagccagcagtttccagatatggggattgatccccttcagtgcctcttgggacatggaatattggcagactctctctggatctgctcctggattgagctctacaaatacaggagggtggagctgggagaggggtagtgttgatgttagacacataatgtgacatcagcctgctctgcatctgtgtctccagaggaacttgaacttcccagctgtactttggtaattgcttctgaaaaaaacctcctggccacacagaaatcaaataaaatttttaatcaagcaaattgaaaaatccaggctgtattataaacatcaattaaaagaagatctgggcctgggccttttgatgtttcctgaccagcttggatgctgtcacctttgtctactcagaagggagtaaaggcagtctaaagtcatcctcgccatcctcgccttcctggtgtgattcgcagaagtagaaagaacaccagcagctgctttgaaataccctggagctggcagatgatggacaggaaacagacagctgagaccctgctgaccctgtctcctgctgaagttgccaacctcaaggaagggatcaatttttttcgaaataagactactgggaaagagtacattttatacaaggagaaggaccatctaaaggcatgcaagaacctctgcaagcaccagggaggcctgttcatgaaagacatcgaggatttagatggaaggtccgttaaatgcacaaagcacaactggaagttagacgtgagcaccatgaaatatatcaaccctccagggagcttctgtcaagacgagctcgttattgaaatggatgaaaacaatgggctttccctggtagaactgaaccctcctaacccctgggactctgatcccaggtctcctgaagaattagcttttggggaagtacagataacatatctcactcatgcctgcatggacctcaagttgggagacaagcgaatggtatttgacccttggttaattggccctgcttttgcccgaggatggtggttgctacatgagcctccatctgactggttggagaggctgtgcaaagcagacctcatttatatcagccacatgcactcagaccacctgagctaccctaccctgaagcagctttcccagagacgaccagacattcccatttatgttggcgacacagaaaggcctgtgttttggaacctggatcagagtggcgtcgggttaactaacatcaacgtggttccatttggaatatggcaacaggtagacaaaagtctgcggttcatgatcttgatggacggcgttcatcctgagatggacacatgcattatcgtggagtacaaaggtcataaaatactcaacacagtggactgcaccagacccaatgggggaaggcttcctgagaaagttgctctaatgatgagtgatttcgcaggaggtgcatcaggctttccaatgactttcagtggtggaaaatttactgaggaatggaaagcccagttcattaaggctgaaagaagaaagcttctgaattacaaagctcagctggtgaaggacctgcagccccgaatctactgtccgtttgctgggtactttgtggagtctcacccatctgacaagtacattaaggaaacaaacaccaaaaatgacccaaatcagctcaacaatcttatcaggaaaaactctgacgtggtgacatggaccccacgacctggcgctgtcctcgaccttggcaggatgctgaaggacccaacagacagcaagggcattgtggagcctccagaggggacaaagatttacaaggattcctgggactttggcccgtacctggagatcttgaattctgctgtcagagatgaaatcttctgtcattcatcctggattaaagagtacttcacgtgggctggatttaagaattacaacctggtggtcaggatgattgaaacagatgaagatttcagcccttttcctggagggtacgactatctggtggactttctagatttatcctttccgaaagaaagacccagccgggagcatccttatgaagaaatccatagccgggtggatgtcatcaggtacgtggtgaagaacggcctgctgtgggatgatctgtatattggattccagacccgattgctgcgggaccctgatatataccatcatctgttttggaatcattttcagataaaactccctctaacaccacccaactggaagtcgttcctaatgcactgtgattagtctggacctggtaagtcccaggaccccagcccagaggatggtgcctgaacattcaagatgggtctcccctgcttcgataaacctttctgga

SEQ ID NO: 15 - DNA, 1165 bp, Mus musculus
  CDS (8)..(1117)
aagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacg

(19) United States
(12) Patent Application Publication
(10) Pub. No.: US 2006/0147429 A1
(43) Pub. Date: Jul. 6, 2006

(54) FACILITATED CELLULAR RECONSTITUTION OF ORGANS AND TISSUES

(76) Inventor: Paul Diamond, Secaucus, NJ (US)

Correspondence Address: PAUL DIAMOND, 942 SCHOPMANN DRIVE, NO. 2, SECAUCUS, NJ, 07094, US

(21) Appl. No.: 11/306,414

(22) Filed: Dec. 27, 2005

Related U.S. Application Data
(63) Continuation-in-part of application No. 11/162,715, filed on Sep. 20, 2005.
(60) Provisional application No. 60/597,009, filed on Nov. 3, 2005.
(60) Provisional application No. 60/640,445, filed on Dec. 30, 2004.

(51) Int. Cl. A61K 35/12

(52) U.S. Cl. 424/93.7

(57) ABSTRACT

One aspect of the invention provides improved methods for the production of at least substantially cellularly human solid organs and solid tissues in non-human mammalian hosts. Related aspects of the invention provide: transplantation-based methods for obtaining organ and/or tissue-specific gene expression and/or phenotypes with respect to selected cell types and/or at least substantially all cell types of a solid organ or tissue; methods for cellularly reconstituting solid organs and tissues with replacement cells, such as human cells, with respect to selected cell types and/or at least substantially all cell types of an organ or tissue; modified non-human mammals for cellularly reconstituting solid organs and tissues with replacement cells; and the cellularly reconstituted solid organs and tissues. The production of human organs and tissues according to the invention overcomes the limitations associated with xenotransplantation of animal organs and tissues to humans.

### CROSS-REFERENCE TO RELATED APPLICATIONS

This application is a continuation-in-part of U.S. application Ser. No. 11/162,715 filed Sep. 20, 2005 and claims the benefit of U.S. provisional application Ser. Nos. 60/597,009 filed Nov. 3, 2005 and 60/640,445 filed Dec. 30, 2004, each of which is incorporated by reference herein in its entirety.

### FIELD OF THE INVENTION

The invention relates to the fields of tissue engineering and transplant biology.

### BACKGROUND

International Application No. PCT/US2003/029251 (Publication No. WO 2004/027029 A2) to Beschorner et al. discloses a method for the production of chimeric mammalian organs in which the incorporation of foreign replacement cells into a target organ of a fetal mammal supported by a pregnant female is facilitated by the selective killing of cells in the fetal target organ in response to a set of conditions, while the same conditions do not kill cells of the corresponding organ of the pregnant female. In this manner, biological functions such as liver function can be disrupted in the fetus to facilitate cellular replacement while the animal carrying the fetus is not injured and continues to support the fetus and compensate for the loss of functions in the fetus.

Rhim et. al., Proc. Natl. Acad. Sci. USA, Vol. 92, 4942-46 (1995) discloses the selective repopulation of the hepatocytic cell content of a mouse liver by normal xenogeneic transplanted adult hepatocytes, in transgenic mice characterized by a defect in hepatic growth potential and function

### SUMMARY

The invention provides methods for the cellular reconstitution of organs and tissues with replacement cells of a selected species. In one aspect, the invention provides improved methods for the production of chimeric human, non-human organs and tissues in non-human mammalian hosts and for the production of at least substantially completely human organs and tissues in non-human mammalian hosts.

One aspect of the invention provides a method for reconstituting a donor organ with replacement cells, that includes the steps of: transplanting a donor organ or tissue, such as a solid organ or tissue, or part thereof from a human or non-human mammal donor to a non-human mammal host that supports the donor organ or tissue in a living state, wherein, at least part or at least substantially all, of the cells of the donor organ or tissue are at least substantially selectively killable; selectively killing at least some of the donor organ or tissue cells; and introducing replacement cells into the donor organ or tissue to replace endogenous donor cells of the organ or tissue. In one variation, the donor organ or tissue expresses a suicide gene under control of a broad-activity promoter so that the donor cells are thereby killable in response to a set of one or more conditions, the cells of the non-human host mammal and the replacement cells are not killable in response to the set of one or more conditions that kills the donor cells, and the step of selectively killing at least some of the donor cells comprises application of the set of one or more conditions.

In another variation, the step of selectively killing the donor cells is performed after the donor organ or tissue is transplanted into the host. The step of introducing replacement cells into the donor organ or tissue may be performed before and/or after transplanting the donor organ or tissue into the host. In one embodiment, the replacement cells are introduced into donor mammal where they incorporate into the donor organ or tissue.

A related aspect of the invention provides a method for reconstituting a donor organ with replacement cells, that includes the steps of: transplanting a solid organ, such as a kidney, lung, heart, liver, and pancreas, or solid tissue or solid part thereof from a human or non-human mammal donor into a non-human mammal host that supports the donor organ or tissue or part thereof in a living state; selectively killing at least some of the native donor organ or tissue cells after the donor organ or tissue is transplanted into the host; and introducing replacement cells, such as human replacement cells, into the donor organ or tissue or part thereof, before and/or after the organ or tissue or part thereof is transplanted to the host, the replacement cells being capable of replacing at least some of the endogenous cells or cell types of the donor organ or tissue. Cellular reconstitution of the organ or tissue or part thereof by the replacement cells is thereby promoted. In one variation, the donor is a non-human mammal and the step of introducing replacement cells into the donor organ or tissue includes introducing stem cells into the donor mammal during a fetal or neo-natal stage of development so that the solid organ or solid tissue or solid part thereof includes stem cell-derived replacement cells already incorporated therein before it is transplanted to the non-human host mammal. In another variation, the donor organ or tissue expresses a suicide gene under control of a broad-activity promoter so that the donor cells are thereby killable in response to a set of one or more conditions, the cells of the non-human host mammal and the replacement cells are not substantially killable in response to the set of one or more conditions that kills the donor cells, and the step of selectively killing at least some of the donor cells comprises application of the set of one or more conditions.

Another aspect of the invention provides a method for reconstituting a donor organ with replacement cells, that includes the steps of: transplanting a solid organ or solid tissue or solid part thereof from a human or non-human mammal donor to a non-human mammal host that supports the donor organ or tissue or part thereof in a living state, wherein the growth of at least some or at least substantially all of the cells of the donor organ or tissue is selectively impairable in response to a set of one or more conditions, and wherein the growth of the endogenous host cells and the replacement cells is not substantially impairable by the set of one or more conditions; selectively impairing the growth of at least some of the donor organ or tissue cells after the organ or tissue is transplanted into the host by applying the set of one or more conditions; and introducing replacement cells, such as human replacement cells into the donor organ or tissue or part thereof, before and/or after transplantation of the organ or tissue or part thereof to the host.

A further aspect of the invention provides a method for obtaining a non-human animal, such as a non-human mammal, wherein a desired trait is limited to a preselected organ or tissue or part thereof supported by the animal, that includes the steps of: transplanting a preselected solid organ or solid tissue or solid part thereof from a human donor or a non-human animal donor, such as a non-human mammal, to a non-human animal host, such as a non-human mammal host, wherein the solid organ or solid tissue or solid part thereof is supported in a living state, wherein at least some of the endogenous cells types of the transplanted donor solid organ or solid tissue or solid part thereof have a desired trait, and wherein the endogenous cells of the host animal at least substantially do not express the trait. The corresponding organ(s) or tissue(s) of the host may be left in place or may be at least partially removed. In one variation, the donor and host are each non-human animals of the same species, such as non-human mammals of the same species. In another variation, the trait comprises inducible or constitutive expression of a negative selection marker. In a more specific embodiment, the trait includes developmentally regulated expression of a negative selection marker. In another variation, the trait comprises susceptibility to cell-death or growth-impairment in response to a set of one or more known or preselected conditions. A related variation further includes the step of providing the set of conditions that cause cell the death or growth-impairment. In a different variation, the trait includes the inducible or constitutive or developmentally regulated expression of a preselected transgene that is not present, or at least substantially not present, in a functional form in the genome of the host animal. The expression of the transgene may, for example, be put under the control of a broad-activity promoter or one or more cell type-specific promoters. In one embodiment, the preselected transgene is a preselected suicide gene or a preselected growth-impairing gene.

Another aspect of the invention provides a non-human host animal, that includes a living non-human mammal host; at least part of a solid organ or solid tissue transplanted from a human or non-human donor mammal, supported in a living state by the host; and replacement cells, such as human replacement cells, present in the at least part of the organ or tissue from the donor mammal. In one variation, at least some of the donor mammal cells of the at least part of the organ or tissue transplanted from the donor are selectively killable or growth-impairable versus the endogenous host cells and the replacement cells.

Still another aspect of the invention provides a method for reconstituting an organ or tissue with replacement cells, that includes the steps of: providing at least one non-human mammal embryo or fetus that is heterozygous or homozygous for a transgene conferring organ or tissue specific expression of a suicide gene or any sort of negative selection marker (trait), or otherwise having organ or tissue-specific expression of a negative selection marker; and transferring the at least one embryo or fetus to a surrogate mother animal (not the genetic parent of the embryo or fetus) of the same or a different species that at least substantially does not express the transgene, for example, as a result of lacking a functional copy thereof, or at least substantially does not express the negative selection marker, the transferred embryo or fetus being supported in a living state by the surrogate mother and continuing development within the surrogate mother. One variation includes a further step of: during a fetal stage of development of the embryo or fetus, introducing replacement cells, such as human replacement cells, directly or indirectly into the organ or tissue of the fetus. In one variation, the replacement cells are introduced into a fetus before the fetus is transplanted to a surrogate mother or upon the transplantation. In another variation, the replacement cells are introduced into a fetus (that was transferred to the surrogate mother or that is derived from a transferred embryo) after the fetus is established within the surrogate mother. The step of incorporating replacement cells into the organ or tissue may include introducing stem cells, such as human stem cells, into the fetus. Endogenous cells of the organ or tissue of fetus may then be selectively killed or growth-impaired to promote their replacement by the replacement cells.

A related aspect of the invention provides a non-human surrogate animal, that includes: a non-human mammal surrogate animal; and a non-human mammal embryo or fetus which is supported by the surrogate but which is not the progeny of the surrogate, wherein the embryo or fetus is heterozygous or homozygous for a trait that imparts the ability to selectively kill or growth impair at least some cells of a preselected organ or tissue in response to a set of one or more conditions, and wherein the surrogate lacks the trait, so that subjecting the surrogate having the transferred fetus or a fetus derived from the transferred embryo supported therein to the set of one or more conditions kills or growth impairs cells of the preselected organ or tissue in the fetus and at least substantially does not kill or growth impair corresponding cells in the host animal. In one variation the transferred fetus or a fetus derived from the transferred embryo includes replacement cells, such as human replacement cells, present in the preselected organ or tissue, wherein the replacement cells are at least substantially not killable or growth impairable by the set of one or more conditions.

The invention also provides methods for culturing xenogeneic cells, in any form (e.g. a solid or distributed, chimeric or non-chimeric organ or tissue or part of a body, stem cells, etc.) in a non-human mammal host in which the transfer of one or more xenoantigens from the host to the hosted xenogeneic cells is reduced and/or in which the transfer of one or more tolerance-promoting biomolecules from the host to the hosted cells is promoted.

### BRIEF DESCRIPTION OF THE DRAWINGS

FIGS. 1A, 1B and 1C are schematic representations of transgene constructs that can be used to produce transgenic animals expressing suicide genes.

### DETAILED DESCRIPTION

The invention provides, in part: transplantation-based methods for obtaining organ and/or tissue-specific gene expression and/or phenotypes for solid organs and tissues in a host animal, with respect to selected cell types and/or at least substantially all cell types of an organ or tissue; methods for cellularly reconstituting solid organs and tissues with replacement cells, such as human cells, for selected cell types and/or at least substantially all cell types of an organ or tissue; modified non-human mammals for cellularly reconstituting solid organs and tissues with replacement cells; and the cellularly reconstituted solid organs and tissues.

According to one aspect of the invention, a selected donor organ and/or tissue from a donor individual that include cells that can be killed or growth-impaired in response to a set of conditions is surgically transplanted to a non-human host individual whose cells are not killable or growth-impaired by the same set of conditions. The transplanted donor organ or tissue is supported in a living state by the host. In this manner, a chimeric animal in which at least some of the cells of selected donor organs or tissues can be selectively killed or growth impaired is provided. In one embodiment, the donor individual and the host individual are both mammals. According to another aspect of the invention, a chimeric animal of this sort can be used to prepare organs and tissues that are at least partially reconstituted by foreign replacement cells in the following manner. In one embodiment, the donor organ or tissue is a solid organ, a solid tissue or a solid part or section of a solid organ or solid tissue. The tissue may be a simple tissue or a complex tissue.

Foreign replacement cells capable of replacing the cells of the donor organ or tissue can be introduced directly or indirectly into the donor tissue, before and/or after the donor organ or tissue is transplanted into the non-human animal host. The engraftment and growth of the foreign replacement cells in the donor organ or tissue is facilitated by treating the donor organ or tissue with the set of conditions that kills or impairs its cells. Such treatment can be provided before and/or after the donor organ or tissue has been transplanted to the host animal. In one embodiment, the donor individual and host individual are each mammals and the foreign replacement cells are also mammalian. In a related embodiment, the donor individual is a non-human mammal or a human being, the host individual is a non-human mammal, and the foreign replacement cells are human cells. In another embodiment, the donor and host individuals are each non-human mammals and the foreign replacement cells are human cells.

In a related aspect of the invention, a donor organ and/or tissue from a donor individual that includes native cells that are growth-impaired (or impairable) is surgically transplanted to a second non-human host individual whose cells are not growth-impaired (or not similarly impairable). Again, the transplanted donor organ or tissue is supported in a living state by the host. A chimeric animal of this sort may be used to prepare organs and tissues that are at least partially repopulated by foreign replacement cells that have a populative advantage versus the growth-impaired donor organ or tissue cells.

One aspect of the invention limits the effect of cell death-promoting or growth-retarding transgenes and/or genetic modifications (spontaneous or engineered) or susceptibilities to selected donor organs and tissues or part thereof in a non-human mammal host by transplanting a selected organ or tissue or part thereof having such a transgene or genetic modification or susceptibility from a donor, such as a non-human mammal or a human, to a non-human host, such as a non-human mammal host, not having the same such transgenes or genetic modifications or susceptibility, so that the specific effect of the transgenes or genetic modifications or susceptibility of the donor will only be felt upon the donor organ or tissue in the host.

For example, in a first strategy according to the invention, non-human transgenic donor organisms can be provided that express a suicide gene or any other sort of negative selection marker or system that allows the donor organ or tissue cells to be conditionally and at least substantially selectively deleted (killed) versus the host cells. In a second strategy according to the invention, the donor organ or tissue is characterized by a susceptibility, for example, to a chemical agent that kills cells, which susceptibility the host lacks, for example, as a result of a genetic modification. This strategy can be used with any type of donor and host but is particularly useful when the donor is a human being since, as general matter, a functionally developed donor organ or tissue from a person cannot be stably genetically modified on a large scale with a negative selection marker. For example, a transgenic mammal host, such as a pig, can be produced that generally expresses the neo^{R} (neomycin/kanamycin resistance) gene, which confers resistance to the mammalian cell selection agent G418 (Geneticin). A selected human donor organ or tissue is transplanted to the mammal host where it is supported in a living state by the host. Since unmodified human cells are susceptible to G418, cell death can be selectively induced across the cell types of the human donor organ or tissue by administering G418 to the host animal following transplantation to the host and/or before the human organ or tissue is placed within the host. Where the goal is the progressive reconstitution of the human organ or tissue with replacement cells, the replacement cells whether human or non-human can be genetically modified to also be resistant to the selection agent so that engrafted replacement cells are not killed during multiple rounds of administering the selection agent. In practice, irrespective of which of the aforementioned strategies is used, care should be taken to avoid administering a selection agent or otherwise inducing selection to such an extent that total necrosis of the donor organ or tissue occurs as the graft could be lost. Appropriate dosage regimens for particular organisms, organ/tissue types and selection mechanisms can be determined empirically by a process of routine experimentation.

Several advantages are provided by various aspects of the invention, including but not limited to the following.

First, since cells of the corresponding host organ or tissue are not deleteriously affected to a substantial extent, they can continue to function and support the host, despite the deletion or impairment of donor organ cells or donor tissue cells. This is particularly apparent in the case when the donor organ or tissue does not replace or does not entirely replace the corresponding organ or tissue in the host. This may be achieved according to the invention by heterotopic transplantation (without removing the corresponding host organ or without removing all of it) of the donor organ or tissue to the host, or hemiorthotopic transplantation where one paired organ of the host (e.g., a lung or a kidney in a mammal) is replaced by a corresponding organ of the donor while the other one of the pair remains in the host.

Hence, in contrast to Beschorner et al., the present invention does not require that the recipient mammal for foreign replacement cells is a fetus supported by a pregnant female. Instead, according to the mammalian embodiments of the present invention, the host mammal that carries the mammalian donor organ or tissue that includes or will include replacement cells can be at a fetal stage or at a post-birth stage of development.

Second, according to one aspect of the invention, for a given organ or tissue, a more complete selective deletion and/or growth impairment against a broader spectrum of cell types, and even at least substantially all of the cell types, constituting the donor organ or tissue, can be achieved without requiring the identification of or use of organ or tissue or cell-type specific promoters in order to obtain expression in each cell type constituting a donor organ or tissue. Many organs and tissues are complex, consisting of more than one or several different cell types. This advantage is illustrated in the following embodiment.

In one embodiment, a transgenic non-human donor mammal having a cell death promoting suicide gene under the control of a constitutive or inducible, broad-activity promoter (active in many or at least substantially all cell types of an organ or tissue of interest or even of the entire organism, on a constitutive basis or upon induction) so that cells die in response to a set of conditions is provided. A non-human mammal host in which at least substantially all of the cells are not deletable in response to this set of conditions is also provided. A selected organ or tissue is transplanted from the donor to the host, where it is supported in a living state by the host. When it is desired, a plurality of cell types in the transplanted donor organ or tissue can be deleted by providing the host with the set of conditions to which the donor cells are sensitive, while the corresponding host cells and/or the host cells in general are not deleteriously affected. Replacement cells can be introduced into the host that comprises the donor organ or tissue before, after and/or during the deletion of the donor cells and in any manner that results in the localization of at least some of the replacement cells to the donor organ or tissue. In this manner, the replacement of a broad range or at least substantially all of the cell types that constitute the donor organ or tissue by the replacement cells is facilitated, while the endogenous organs and tissues of the host, for example vascular endothelium and/or endothelium generally, are not deleteriously effected.

A combination of different cell type specific promoters for a subject organ or tissue, can also be used to facilitate the replacement of varying cell types with replacement cells in a subject organ or tissue. Where such promoters are differentially inducible or drive the expression of different types of suicide genes or negative selection markers, conditional cell deletion of each of the subject cell types can be performed simultaneously or in a staggered, progressive manner. A combination of a broad-activity promoter and at least one cell type specific promoter can be also used to obtain a temporally staggered and specific replacement, e.g., of at least substantially all of the cells of a subject donor organ or tissue, with foreign replacement cells. In this case, deletion using the tissue-specific promoter(s) can be performed before deletion based on the broad-activity promoter. The following examples illustrate how this can be achieved, according to the invention.

Example—Different Suicide Genes

A transgenic non-human donor mammal, such as an ungulate, is provided in which a first tissue cell-type specific promoter drives the expression of a first suicide gene, whereby cells expressing the suicide gene are killed under a first set of conditions and in which a second, broad-activity (constitutive or inducible) promoter drives the expression of a second, different suicide gene, whereby cells expressing the second suicide gene are killed by a second set of conditions that is different than the first set of conditions. The cell-type specific promoter used will be active in at least one cell-type present in a tissue or organ that will be engineered with replacement cells. For example, the first promoter can be a mammalian Albumin promoter to drive expression of the first suicide gene in hepatocytic cells of the liver. The organ or tissue, such as the liver, is then transplanted to a non-human mammal host, such as another ungulate of the same or a different species, whose cells are at least substantially not deletable by the first or second set of conditions, for example, as a result of not having the same promoter-suicide gene constructs of the donor mammal. In a first replacement procedure, the host mammal is then provided with the first set of conditions to kill cells of the transplanted organ in which the first suicide gene is expressed under control of the first promoter and a first set of replacement cells capable of replacing the killed cells are directly or indirectly provided to the transplant for engraftment and replacement therein. The first set of conditions may optionally be continued on an intermittent or continuous basis to further facilitate replacement of the cells expressing the first suicide gene by the first set of replacement cells. In a second replacement procedure, the second set of conditions is provided to the mammal host to kill the remaining cells of the transplant that originate from the donor and a second set of foreign replacement cells capable of replacing these remaining cells is directly or indirectly provided into the transplant. Again, the second set of conditions can continue to be provided on an intermittent or continuous basis to further facilitate the engraftment of and replacement by the second set of replacement cells.

Optionally, a non-human host mammal may also be transgenic, for example, so that its cells are selectively deletable over the donor cells and over the foreign replacement cells in response to a third set of conditions, which is different than the first and second sets of conditions. This can be achieved, for example, by expressing a third suicide gene in the host mammal that is different from the first and second suicide genes. In this manner, the organ or tissue product that is obtained by engraftment and replacement of at least some donor mammal cells can be cleared of unwanted host mammal cells that may be present within the organ or tissue.

The process of clearing the product organ or tissue of any donor mammal cells can be performed while the product organ or transplant is in the mammal host and/or after it has been removed from the mammal host.

Example—Liver

A more specific variation of the previous example relating to providing a chimeric non-human, human liver or an at least substantially, fully cellularly human liver is provided for further illustration.

A transgenic pig donor, is provided in which a first, hepatocytic cell-specific promoter (constitutive or inducible), such as the Albumin promoter drives the expression of a first suicide gene, whereby cells expressing the suicide gene are killed under a first set of conditions and in which a second, broad-activity promoter (constitutive or inducible) drives the expression of a second, different suicide gene, whereby cells expressing the second suicide gene are killed by a second set of conditions that is different than the first set of conditions. Alternatively, instead of a suicide gene, a gene imparting replicative deficiencies, such as the urokinase-type plasminogen activator (uPA) can be put under the control of the Albumin promoter so that replacement cells have a selective repopulative advantage versus the hepatocytic cells expressing uPA.

At least part of the liver of the transgenic donor pig is then transplanted, e.g., heterotopically or parallelotopically, to a host pig whose cells are at least substantially not deletable by the first or second set of conditions, for example, as a result of not having the same promoter-suicide gene constructs of the donor pig. In a first replacement procedure, the host mammal is then provided with the first set of conditions to kill hepatocytic cells of the transplanted liver and a first set of human replacement cells capable of replacing the killed cells, such as human hepatocytes or human hepatocyte progenitor cells are directly or indirectly provided to the donor liver for engraftment and replacement therein. The human replacement cells are also not deletable by the first or second set of conditions. Further, the human replacement cells may be non-transgenic or transgenic. The first set of conditions may optionally be continued on an intermittent or continuous basis to further facilitate replacement of the donor liver hepatocytes by the first set of human replacement cells.

In a second replacement procedure, the second set of conditions is provided to the pig host to kill the remaining pig cells of the donor liver and a second set of human replacement cells capable of replacing these remaining donor cells is directly or indirectly provided into the transplant. Pig cells remaining after the first replacement procedure will be largely be non-hepatocytic cells of the liver in which the Albumin promoter does not substantially drive expression of the first suicide gene such as biliary duct cells and portal tract vessel cells. Again, the second set of conditions can continue to be provided on an intermittent or continuous basis to further facilitate replacement by the second set of human replacement cells.

Immunological Tolerance

According to the invention, the host animal is at least substantially immunologically tolerant of the transplanted donor organ or tissue and of the replacement cells. In a related embodiment, the host animal and the donor organ or tissue are, collectively, at least substantially immunologically tolerant of the replacements cells that are introduced into the system. The invention is not limited to the manner by which a suitably tolerant host animal is obtained or provided and any method or combinations of methods can be used.

One manner in which a host tolerant of a donor organ or tissue can be obtained is by using host and donor animals that are of or derived from the same or closely related varieties of the same species. In a related embodiment, the host and donor animals are of or derived from the same inbred line, or clonally related, so that the host and donor animals are at least substantially congenic. The establishment of inbred lines and methods for cloning are well established for non-human mammals, for example, rodents and ungulates. Host-specific genetic and/or donor-specific genetic modifications to otherwise at least substantially congenic host and donor animals, such as the expression of suicide genes or resistance genes, will generally not affect the tolerance of the host toward donor cells and are also considered at least substantially congenic herein. In the rare event that expression of a donor-specific transgene negatively impacts the tolerance of the host toward donor cells in the absent of other measures, the host can be conditioned to be tolerant to the transgene product, for example, by introducing the transgene product or at least the problematic epitopes thereof to the host during early development, such as during fetal development for mammals.

One method provides a host suitably tolerant of human tissue by using a fetal, non-human, mammal host, such as a fetal pig or sheep or rodent (mouse, rat, guinea pig, capybara, etc.), since the fetal mammalian environment is tolerant to foreign human cells and tissue. In this embodiment, the fetal non-human mammal is the host animal, but it should be understood that such a host can, according to the invention, continue to support donor and/or replacement cells following birth.

Another method provides a host suitably tolerant of human tissue by providing a post-birth-stage, non-human mammal host, such as a pig, that was contacted during fetal development with cells from, or cellular antigens characteristic of, the type of donor that will be used (in order to establish tolerance) and/or, similarly, replacement cells or antigenically similar cells or cellular antigens characteristic of the replacement cells that will be used. In one method, tolerance to foreign cells is imparted in a mammal host by infusing bone marrow cells from the actual donor or same type of donor mammal (to impart tolerance toward donor cells) and/or from a mammal from which the replacement cells will be derived or one that is at least antigenically similar to the intended replacement cells (to impart tolerance toward the replacement cells), into a fetal non-human mammal where they may engraft. Following birth, such an animal has improved tolerance toward human tissue/cells.

A further method provides a non-human host at a post-birth stage of development that is suitably tolerant of donor and/or replacement cells by depleting the immune system of the host using chemical treatment and/or irradiation. Optionally, the ablated bone marrow of the host can be replaced with bone marrow of the type of donor animal to be used and/or of the type of organism, such as human or non-human mammal, from which the replacement cells are or will be derived. For example, x-ray or gamma-radiation, sufficient to destroy at least substantially all of the host's bone marrow can be employed. Such methods are disclosed, for example, in U.S. Pat. No. 6,018,096, which is incorporated by reference herein in its entirety. Chemical ablation, with or without radiation, of at least substantially all of the bone marrow of the animal host using myeloablative agents such as cyclophosphamide, busulfan or combinations thereof can also be employed to obtain substantial tolerance to human tissue.

Another method provides a non-human mammal host suitably tolerant of donor and/or replacement cells by providing a host conditioned according the method of U.S. Pat. No. 6,296,846, which is incorporated by reference herein in its entirety.

Another method provides a host suitably tolerant of donor and/or replacement cells by providing a host that is genetically immunocompromised. Such a host may comprise at least one genetic modification or mutation, intentionally introduced (e.g., targeted) or otherwise arising at any time in the past or in any previous generation, that disrupts the host's immune system. For example, non-human mammals homozygous for mutations in the Rag1 gene (recombination activating gene 1) are characterized by a deficiency in both T-cells and B-cells. Rag1 gene-deficient mice, obtained by knockout methods, have been previously described and are well known in the art (Mombaerts, P. et al., RAG-1-deficient mice have no mature B and T lymphocytes Cell, (1992) 68 (5), 869-77, which is incorporated by reference herein in its entirety). Swine genetically modified to be deficient in the Rag1 gene are disclosed in U.S. Pub. No. 20050155094 (application Ser. No. 10/503,464), which is incorporated by reference herein in its entirety. Non-human mammals homozygous for mutations in the Prkdc gene, known in the art as a type of SCID (severe combined immune deficiency), are also characterized by a deficiency in both T-cells and B-cells. Non-human mammals homozygous for mutations in the Foxn1 gene, known in the art as nude mammals, are characterized by thymic dysgenesis with deficiency in T-cells and partial defects in B-cell development. All three of these mutants are also characterized by secondary immune defects relating, for example, to antigen presenting cells (APCs) and natural killer cells (NK cells).

Non-human mammals having further mutations in the lnterleukin-2 Receptor γ Chain and/or in β₂-microglobulin may also be used. For example, SCID/IL2 Receptor γ Chain^{null} or SCID/β₂-microglobulin^{null} non-human mammals may be used as hosts and/or donors. See Ishikawa et al., Development of functional human blood and immune systems in NOD/SCID/IL2 receptor {gamma} chain(null) mice Blood (2005) 106(5), 1565-1573, and Yoshida et al., Human cord blood-derived cells generate insulin producing cells in vivo, Stem Cells 2005 23(9): 1409-1416, each of which is incorporated by reference herein in its entirety NOD/SCID/IL2 Receptor γ Chain^{null} mice are available for purchase from The Jackson Laboratory (Bar Harbor, Me.) as Stock #004048 subject to the consent of the developer. NOD/Cg-Prkdc^{scid} B2m^{tm1Unc/J} mice are available for purchase from The Jackson Laboratory as Stock #002570. Each of these lines is characterized by tolerance to human cells and excellent engraftment and multi-lineage development of human cells therein. For example, in Ishikawa et al. (2005), 1×10⁵ Lin⁻ hCD34⁺ cells or 2×10⁴ Lin⁻hCD34⁺hCD38⁻ cells were transplanted into irradiated (100 cGy) GGTA1^{null}/NOD/SCID/IL2rγ^{null} via a facial vein within 48 hrs of birth and gave rise to persistent, multi-lineage hematopoiesis. An mRNA sequence of the mouse interleukin-2 receptor gamma chain has been reported as Genbank accession no. D13821 [SEQ ID NO: 15]. Further, as reported in Yoshida et al. (2005), human cord blood cells transplanted into NOD/SCID/β₂-microglobulin^{null} mice within 48 hours of birth can give rise to human, insulin-producing cells therein.

For embodiments in which a non-human mammal donor is used, the donor may optionally be made tolerant to the replacement cells, for example, by any of the methods described herein, in order to prevent or decrease the possibility that donor immune cells, within the donor or that may be transferred to the host animal with a donor organ or tissue, will attack the introduced replacement cells. For example, a donor that is immunodeficient as a result of physical, chemical and/or genetic techniques can be used. Alternatively, a donor that is not severely immunodeficient but is made tolerant toward the replacement cells can be used. Again, a method of introducing bone marrow cells from the organism (or the type of organism) from which the replacement cells are derived into the donor where they may engraft can, for example, be used to condition the donor immune system to tolerate the replacement cells including after the donor organ or tissue has been transplanted to the host.

A genetically modified, conditionally immunodeficient non-human mammal can also be used as a suitably tolerant host animal (or donor animal) pursuant to conditionally inducing the immunodeficiency. For example transgenic mammals including a transgene construct in which expression of a protoxin-to-toxin converting enzyme type of suicide gene is under control of a lymphocyte specific promoter, such as the jak3 (Janus kinase 3) promoter, can be produced. T-cell and B-cell deficiency is conditionally induced by providing the transgenic animal with the protoxin (prodrug). The production of such a mammal is, for example, further provided by International Pub. No. WO 2004/027029 A2 and its corresponding U.S. national phase, application Ser. No. 10/527,587, each of which is incorporated by reference herein in its entirety.

Another manner in which a non-human mammal host at least substantially tolerant of human cells can be provided is by transplanting the human donor organ or tissue to an immunologically privileged site in the host. Reported immune privileged sites include, for example, the testes, the eye (anterior chamber, cornea, and retina), the brain and the placenta. It has also been reported that xenogeneic tissue transplantations under a non-human mammal host's kidney capsule can, at least in some cases, avoid rejection.

Transplantation

A donor organ or tissue, such as that from a human or a non-human mammal, supported by an animal host according to the invention can be maintained in a living state for any number of days. De novo and/or progressive reconstitution of the donor organ or tissue with replacement cells can take place during any part of this time. Moreover, the reconstituted organ or tissue can continue to be maintained in a living state in the host animal. It can also be transferred to a further host animal if desired. The reconstituted donor organ or tissue may be removed, i.e., explanted, from a non-human host when it is needed for a purpose such as experimentation, further processing, transplantation to another non-human host, and/or transplantation to a human being.

The surgical techniques required for the transplantation of various organs and tissues from one individual to another, for example, from pig to pig, from human to human, from pig to sheep, from pig to primate, or from primate to pig, such as from a human to a pig are well developed. According to the invention, an organ or tissue from a human or non-human mammal donor may be transplanted to any suitable location of a non-human mammal host where it can be supported in a living state by the host. Blood supply to vascularized organs and tissues or parts thereof can be established, for example, by anastomosing host and donor arterial vessels to each other and, if required, host and donor venous vessels to each other. Vascular grafts from the donor or host can also be used, if needed, to provide inflow and outflow of blood to the donor organ or tissue in the host. For smaller donor organs such as endocrine glands or thinner donor tissues such as skin, a sufficient blood supply can, for example, be established over a short period by placing the organ or tissue in contact with a vascularized site or surface of the host mammal.

Transplantations of donor organs or tissues, which will be at least partly reconstituted with replacement cells according to the invention, can be performed in an orthotopic, hemi-orthotopic, parallelotopic, or heterotopic manner. An orthotopic transplant, as defined herein, is one in which the donor organ or tissue replaces at least one of the same or homologous structures in the host. A hemi-orthotopic transplant, as defined herein, is one in which the donor organ or tissue replaces one of a pair of the same or homologous structures in the host. When the donor and the host are of the same species or type, the functional anatomy between the donor and host will be the same or at least substantially the same.

A parallelotopic transplant, as defined herein, is one in which the donor organ or tissue is transplanted so that it receives blood from at least part of the same source of the same or homologous structure in the host. Optionally, the blood drainage of the donor organ or tissue can be to at least one of the same blood vessels as the endogenous host structure or to an at least substantially corresponding vessel. Also optionally, the homologous host organ or tissue that remains in the host can be surgically reduced in size if desired.

A heterotopic transplant, as defined herein, is one in which the donor organ or tissue is transplanted into the host in a location or environment that is not characteristic of the location of the organ or tissue in the donor.

The following examples illustrate surgical techniques for transplanting various human or non-human mammal donor organs and tissues to a non-human mammal host for the purpose of implementing the methods of the invention with the organ or tissue, but do not limit the techniques that may be employed for each.

(1) Kidney. The kidney is a paired organ. It is therefore convenient to excise one kidney from the host and transplant the donor kidney, along with a portion of the attached donor ureter, by anastomosing the renal artery and vein of the donor kidney to the abdominal aorta and inferior vena cava of the host, respectively, or to corresponding structures of the host. The donor ureter can be connected to the host bladder, for example, by implanting it into the bladder via a submucosal tunnel. Both kidneys can also be replaced if desired.

(2) Lung. The lung is a paired organ. It is therefore convenient to excise one lung from the host and transplant a donor lung in its place by anastomosis with a pulmonary artery and a pulmonary vein of the host's heart. This step can be performed, for example, by anastomosing a remaining section of pulmonary artery connected to the donor lung to a section of pulmonary artery remaining connected to the host's heart and similarly connecting donor and host pulmonary vein sections. Generally, the transplanted lung should be ventilated in the host to help preserve its structure and function by directly or indirectly connecting it to the trachea (windpipe) or a corresponding structure of the host. However, non-ventilated lung transplants are also within the scope of the invention. If desired or required, a lobe or portion of the remaining host lung can be removed. Both host lungs can also be entirely replaced if desired.

(3) Heart. In a first method, a donor heart is transplanted orthotopically in the host by excising the host heart and anastomosing all of the necessary major arterial and venous blood vessels of the donor heart to at least substantially corresponding vessels of the host. It is well recognized in the art that the functional anatomy of ungulate hearts, and especially that of porcine hearts, is quite similar to that of the human heart. In a second method, a donor heart is transplanted heterotopically to a non-human mammal host, such a sheep or cow, by anastomosing the aorta of the donor heart to the host carotid artery (e.g., end-to-side) and the pulmonary artery of the donor heart to the host jugular vein (e.g., end-to-side).

(4) Liver. In a first method, a donor liver is transplanted orthotopically by excising the host liver and anastomosing (i.) a remaining portion of the host hepatic artery to a portion of the donor hepatic artery connected to the donor liver, (ii.) a remaining portion of the host portal vein to a portion of the donor portal vein connected to the donor liver, and (iii.) the donor hepatic veins attached to the donor liver to either the host hepatic veins connected to the inferior vena cava or directly to the host inferior vena cava.

In a second method, a donor liver or a part thereof is transplanted parallelotopically with respect to a host liver or a remaining part thereof so that each of the livers receives at least part of the hepatic artery blood and the portal vein blood and each drains directly or indirectly into the inferior vena cava. In a variation, the host portal inflow can be split between the donor and host liver so that the donor liver is provided with intestinal-pancreatic effluent and the host liver with gastric-splenic venous blood. (See, e.g., Lilly et al., Split portal flow in heterotopic hepatic transplantation J Pediatr Surg. June 1975; 10(3): 339-48.) Those skilled in the art will recognize that a variety of auxiliary liver transplantation techniques are known in the art and can be readily adapted for parallelotopic and heterotopic liver transplantation according to the invention.

(5) Pancreas. In one method, a donor pancreas with at least a portion of donor duodenum attached is transplanted to the host while the host pancreas is left in place. The donor pancreatic artery and vein can be joined to the host's iliac artery and vein, respectively. The donor duodenum can be joined to the host's small intestine to allow the exocrine enzymes in the main pancreatic duct to enter. In a second related method, the host pancreas is at least partially removed.

(6) Skin. Human skin may be hair-bearing (most of the body, e.g., the scalp) or non-hair-bearing (e.g., palms of hands and soles of feet). Skin consists of three layers (from outside to inside): the epidermis, the dermis (coreum) and a subcutaneous layer comprising areolar and fatty connective tissue. Hair follicles and associated sebaceous (oil) glands are present in hair-bearing skin. In humans, sweat glands are present in both hair-bearing and non-hair-bearing skin. A section of donor skin, such as human skin, including the epidermis and dermis only or the epidermis, dermis and at least part of the subcutaneous layer can be surgically transplanted to a region of a non-human mammal host where the host skin has been at least partly removed. The transplanted skin can be bandaged to ensure good contact with the prepared region of the host to promote the establishment of circulation.

(7) Bone. The blood vessels of bone are numerous. Those of the compact tissue are derived from a close and dense network of vessels ramifying in the periosteum. From this membrane vessels pass into the minute orifices in the compact tissue, and run through the canals which traverse its substance. The cancellous tissue is supplied in a similar way, but by less numerous and larger vessels, which, perforating the outer compact tissue, are distributed to the cavities of the spongy portion of the bone. In the long bones, numerous apertures may be seen at the ends near the articular surfaces; some of these give passage to the arteries of the larger set of vessels referred to; but the most numerous and largest apertures are for some of the veins of the cancellous tissue, which emerge apart from the arteries. The marrow in the body of a long bone is supplied by one large artery (or sometimes more), which enters the bone at the nutrient foramen (situated in most cases near the center of the body), and perforates obliquely the compact structure. The medullary or nutrient artery, usually accompanied by one or two veins, sends branches upward and downward, which ramify in the medullary membrane, and give twigs to the adjoining canals. The ramifications of this vessel anastomose with the arteries of the cancellous and compact tissues. In most of the flat, and in many of the short spongy bones, one or more large apertures are observed, which transmit to the central parts of the bone vessels corresponding to the nutrient arteries and veins. The veins emerge from the long bones in three places: (1) one or two large veins accompany the artery; (2) numerous large and small veins emerge at the articular extremities; (3) many small veins pass out of the compact substance. In the flat cranial bones the veins are large, very numerous, and run in tortuous canals in the diploic tissue, the sides of the canals being formed by thin lamellae of bone, perforated here and there for the passage of branches from the adjacent cancelli. Gray, Henry. Anatomy of the Human Body. Philadelphia: Lea & Febiger, 1918; Bartleby.com, 2000.

Orthotopic and heterotopic transplantations of various bones or parts thereof to a host may be made by anastomosing the major arteries and veins of the donor bone to suitable arteries and veins of the host. Depending on the size of the graft, the donor bone may be anastomosed under magnification to the host femoral artery and veins, for example in an end-to-side fashion. See, e.g., Lee et al., Use of swine model in transplantation of vascularized skeletal tissue allografts, Transplantation Proc. (1998) 30, 2743-2745, which is incorporated by reference herein in its entirety.

(8) Blood vessels. A donor blood vessel may, for example, be transplanted in an orthotopic or heterotopic manner to a host so that blood flow through the vessel is established in the host.

(9) General sites for heterotopic transplantation of donor organs or tissues include, for example, the kidney capsule, subcutaneous space, and splanchnic vasculature generally. It is well known in the art that the kidney and its capsule provide a highly vascularized environment into which numerous sorts of organs and tissues, or parts thereof, can be heterotopically transplanted and supported in a living state. Subcutaneous transplantation of tissue into a host mammal is also well known in the art. The splanchnic vasculature is recognized as a general site for grafting or otherwise obtaining a circulatory connection between a donor organ or tissue and the host's circulatory system. Accordingly, a donor organ or tissue or part thereof can, for example, be heterotopically transplanted under the kidney capsule of a non-human mammal host, transplanted subcutaneously in the host, or grafted or otherwise connected with a host's splanchnic vasculature, in order be supported in a living state by the host.

Surgical transplant techniques for transplanting human and also non-human mammal organs or tissues into a human being are well established. These techniques are readily adaptable for embodiments of the invention in which a human or non-human mammalian donor organ or tissue that has been hosted in, and at least partially reconstituted by replacement cells in a non-human mammal host is later transplanted to a human being.

In one embodiment of the invention, a donor brain or a substantial part thereof is expressly excluded from the organs and tissues that may be at least substantially cellularly reconstituted with human cells according to the facilitated cellular reconstitution methods of the invention. A related embodiment expressly excludes the at least substantial cellular reconstitution of a donor brain or a substantial part thereof with human neurons.

Replacement Dells and their Introduction into a Donor or Tissue

Replacement cells as referred to herein are cells capable of proliferating in a donor organ or tissue into which they are introduced and at least partially replacing at least some of the mature native donor cell types of the organ or tissue. For example, differentiated cells capable of proliferating and reconstituting an organ or tissue can be used, as well as any kind of immature or undifferentiated cell type that can mature or differentiate in the organ or tissue to replace endogenous mature cells of the organ or tissue. Replacement cells may be of any suitable type including, but not limited to, differentiated cells, progenitor cells, tissue-specific stem cells, multipotent stem cells, and omnipotent stem cells. Thus, it should be understood that the term “replacement cells” as used herein may encompass not only the cells in the state in which they were originally introduced into a donor mammal or its organs or tissues or parts thereof, but also cells derived from the introduced cells by the processes of cell division and/or differentiation and/or dedifferentiation. The replacement cells may be from the same species or from a different species as the donor organ and/or host animal and may be primary cells or cells of a cell line. For example, in one embodiment of the invention, human replacement cells are introduced into a human or non-human mammal donor organ or tissue, before and/or after the organ or tissue is transplanted into a non-human mammal host that supports the organ or tissue in a living state in the host. Foreign replacement cells can be genetically modified or unmodified, for example, transgenic or not transgenic.

Many organs and tissues consist of more than one mature cell type. Certain types of replacement cells are only substantially capable of replacing less than the total number of mature cell types that characteristically constitutes a given organ or tissue. For example, mature hepatocytes used as replacement cells in a donor liver are generally capable of replacing hepatocytic cells of the liver but not the other types of cells, such as biliary cells, constituting the liver. In one embodiment of the invention, for a given organ or tissue, more than one type of replacement cell, at least one having an at least partially non-overlapping potential with respect to another, is introduced into a donor organ or tissue so that more than one cell-type of the tissue can be replaced. Multipotential or omnipotential replacement cells can also be used, when available, to replace more than one cell type of a given donor organ or tissue. For a given organ or tissue, such as the liver, a variety of different types of replacement cells with different scopes of potential are known in the art.

Replacement cells can be introduced into a donor organ or tissue in any manner or combination of manners and may be in any form or combination of forms. For example, primary cells from a human or a non-human mammal provider can be obtained for use as replacement cells in a corresponding donor organ or tissue. The primary cells may be of mixed types as obtained or may be selected out and isolated or mixed as desired. Advantageously, the use of the mixture of cell types present in a replacement cell provider organ or tissue (“provider organ or tissue”) can provide most if not all of the types of replacement cells necessary to at least substantially replace each of the types of cells of the donor organ or tissue. As to the form that the primary cell replacement cells take, in one embodiment, parts of a provider organ or tissue may be surgically transplanted into or surgically combined with the donor organ or tissue. For example one or more plugs of tissue from a provider organ or tissue can be transplanted into a corresponding donor organ or tissue or a section of provider organ or tissue can be interfacially joined, surgically, with a suitably prepared donor organ or tissue so that their internal structures are in contact. In this manner, the histological arrangement of cells in the transplanted parts of the provider tissue is not substantially disturbed. In another example, a patch of provider skin can be transplanted within a patch of donor skin, or a section of a liver with a cut face can be surgically joined to the cut face of a donor liver. In another embodiment, a cell suspension of single cells and/or aggregates is formed from part or all of a provider organ or tissue and the suspension is introduced into the donor organ or tissue, either directly, and/or indirectly, for example, by infusing the suspension into the blood stream of the host animal after the donor organ or tissue is established therein or by infusing the suspension into the blood stream of a donor animal before the donor organ or tissue is transplanted to the host animal. For example, pancreatic islets or a general suspension of pancreas cells from a provider pancreas are infused into a donor pancreas and/or one or more plugs of a provider pancreas are inserted into a donor pancreas. A suspension of provider vascular endothelial or organ endothelial cells can be contacted with, for example by infusion or perfusion, donor organ or tissue endothelium. In another embodiment, replacement cells can be introduced into a fetal or post-birth donor mammal where they integrate into one or more organs or tissues of the donor mammal to form one or more chimeric organs or tissues that include cells of the donor mammal and the replacement cells. In this manner, replacement cells are already present in a donor organ or tissue when it is explanted from the donor mammal. Any suitable techniques such as those employing catheters or needles may be used to directly or indirectly infuse or inject a suspension of single cells and/or cell aggregates into a donor organ or tissue or any desired location of a donor or host.

The following examples illustrate specific types of cells that can also be used as replacement cells for particular donor organs or tissues.

Tissue-based stem cells have the ability to proliferate and differentiate into the corresponding tissue cells. For example, pancreatic duct cells can differentiate into islets of Langerhans. Hepatic oval cells can differentiate into hepatocytes. Adult stem cells and certain tissue-based stem cells are characterized by plasticity, being capable of differentiating into other types of cells. For example, hematopoietic stem cells can differentiate into cells such as endothelial cells, neurons, glia, hepatocytes, cardiomyocytes, renal tubular cells, pulmonary epithelium, intestinal cells, skin epithelium, bone, cartilage, muscle, fat, and brain. Adipose stem cells can also differentiate into a wide variety of cell types. Embryonic stem cells have the ability to proliferate and differentiate into any tissue. Either the embryonic stem cells, cell lines produced from embryonic stem cells, or progenitor cells derived from the embryonic stem cells or cell lines can, for example, be used to reconstitute donor organs or tissues according to the invention. Methods for providing differentiated stem cells are provided, for example, by U.S. Pat. No. 6,576,464. Methods of differentiating human embryonic germ cells are provided, for example, by U.S. Pat. No. 6,562,619.

Bipotential embryonic liver stem cell lines, as shown in the mouse, can also contribute to liver regeneration and differentiate as bile ducts and hepatocytes. Strick-Marchand et al. Proc. Natl. Acad. Sci. USA Jun. 1, 2004, vol. 10, no. 22, 8360-8365. U.S. Pat. No. 6,129,911 also discloses liver stem cells that can be used and is incorporated by reference herein in its entirety.

Myoblasts, which differentiate into smooth muscle, skeletal muscle or cardiac muscle (cardiomyocytes) can be used as replacement cells for muscle tissues. Cardiomyocytes can also be used as replacement cells for heart muscle.

Replacement cell provider animals or cell lines may be genetically modified, for example to impart resistance to preselected selection agents and/or to eliminate xenoantigens. In one embodiment, human replacement cells are genetically engineered to express resistance to a selection agent. Such cells can be used in conjunction with a similarly resistant host and a donor organ or tissue that is susceptible (not resistant) to the selection agent.

Preferably, foreign replacement cells are introduced into the donor organ or tissue in a substantially sterile manner. They may, for example, be introduced before, during or after selectively and conditionally injuring native cells of the donor organ or tissue, provided that the replacement cells survive after the injury occurs. In one embodiment, the replacement cells are introduced into the donor or tissue after it has been explanted from the donor but before it has been transplanted to the host. Optionally, one or more further introductions of replacement cells into donor organ or tissue can be performed after the organ or tissue is established in the host. In another embodiment, replacement cells are introduced into the donor organ or tissue after it has been transplanted to the host. Another embodiment of the invention provides that a donor tissue or organ is selectively injured by a general method, such as radiation exposure, before it is transplanted into the host. In a variation of this embodiment, replacement cells are introduced after the general injury so that they are not harmed by the injury. In a further variation, further selective and conditional deletion of the donor organ or tissue can be performed after it has been transplanted to the host.

Example—Replacement Cells Engrafted in Donor Organ or Tissue after Explantation from the Donor Mammal

A CMV-xTK donor liver from a transgenic pig is transplanted orthotopically into a substantially congenic host pig that does not express xTK and is supported in a living state in the host. Once the donor liver in established in the host, ganciclovir is administered intravenously to cause cell death in the donor liver. Human replacement cells capable of regenerating the liver, such as hepatocytes, liver progenitor cells, or hematopoietic stem cells are then introduced into the donor liver, for example, by infusion via the portal vein and/or by direct injection. The replacement cells engraft into the donor liver. Further rounds of ganciclovir administration facilitate further replacement of the donor liver cells by the replacement cells. In an alternative case, one or more human liver plugs is inserted into the donor liver before it is transplanted into the host. Rounds of ganciclovir administration to the host are again used to facilitate further replacement of the donor liver cells by the replacement cells (originating from the plugs).

In cases where a suspension of replacement cells is introduced, the optimal number of cells introduced depends on the source and can be determined by routine experimentation. For fetal pigs, replacement cells can, for example, be introduced at 52 days gestation or seven days after the prodrug is administered (range 25 to 114 days gestation). A human hepatocyte dose of 5×10⁶/fetus (range 1×10⁵ to 5×10⁷ cells/fetus) can, for example, be used. A liver stem cell dose of 5×10⁵/fetus (range 1 to 5×10⁷/fetus) can, for example, be used. Bone marrow and umbilical cord blood also provide sources of pluripotential progenitor cells that can differentiate into hepatocytes. A human cord blood dose of 2.5×10⁷ nucleated cells/fetus (range 1×10⁶ to 10⁸) can, for example, be used.

In one embodiment of the invention, replacement cells, such as human replacement cells, are introduced into a non-human donor mammal, such as a pig, sheep or rodent (e.g., mouse, rat, guinea pig, capybara, etc.), at a preimmune fetal stage where they incorporate into organ(s) and/or tissue(s) of interest to form chimeric organs and/or tissues that include cells derived from the introduced replacement cells and the donor mammal's own cells. The donor mammal cells are selectively deletable (or can be selectively growth-impaired) versus the replacement cells and versus cells of a host mammal into which the chimeric organ will be transplanted, for example, by virtue of expressing a suicide gene as described herein, or by not expressing a resistance mechanism that is present in the replacement cells and the host mammal, as described herein. In the case where the donor mammal cells express a suicide gene or growth-retarding gene, the regulatory units driving expression of the gene may consist of one or more broad-activity promoters (constitutive or inducible) and/or one or more tissue or cell-type specific regulatory units (constitutive or inducble), as described herein. During the fetal stage or at any stage after birth, the chimeric donor organ or tissue that already includes replacement cells is transplanted to a mammal host, for example, a non-human mammal host, such as by any of the methods described herein, where it is supported in a living state. Once the chimeric donor organ or tissue is established in the host, it can be subjected to one or more rounds of the conditions that selectively delete (or impair) the donor mammal cells in the chimeric organ, thereby facilitating a progressive and selective repopulation of the organ or tissue by the replacement cells that are present in the organ. The selective deletion of the donor cells (versus the replacement cells) from the chimeric donor organ or tissue may, for example, be begun before the donor organ is transplanted to the host mammal and/or immediately on transplantation to the host mammal.

The following example illustrates a variation of the embodiment employing a sheep donor and host and human replacement cells to produce a human cell populated liver.

Example—Replacement Cells Incorporated in Donor Organ or Tissue Before Explantation from the Donor Mammal

A transgenic sheep fetus (or alternatively a transgenic pig fetus) is provided that expresses a suicide gene under control of a constitutive, broad-activity promoter, such as the CMV-xTK transgene, and which has reduced expression of at least one xenoantigen (with respect to human tolerance of xenogeneic material) and/or is transgenic for expression of at least one transferable tolerance-promoting biomolecule, such as hDAF and/or MIRL. The fetus is carried by a pregnant female.

Human cells capable of giving rise to hepatocytes, to other liver cells and/or to hematopoietic cells in the sheep fetus are provided for introduction into the fetus during its preimmune stage. Such human cells include, but are not limited to, fractionated or non-fractionated preparations of adult human bone marrow, umbilical cord blood cells, placental stem cells, and mobilized peripheral blood stem cells. Sheep fetuses are preimmune until about day 77 of gestation. The preparation and transplantation of the replacement cells may, for example, be carried out according to the method of U.S. Pub. No. 20020100065, which is incorporated by reference herein in its entirety. Human bone marrow or cord blood may, for example, be fractionated, such as by magnetic cell separation and/or fluorescence-activated cell sorting, to enrich for selected phenotypes, such as those associated with hematopoietic stem cells, e.g., CD34⁺ Lin⁻ phenotypes. Unfractionated human bone marrow or human umbilical cord blood may, for example, also be used.

Preimmune fetal sheep at 55-60 days of gestation are injected with a replacement cell preparation such as 1×10⁶ to 1×10⁸ (e.g., 2.5×10⁷) nucleated human cord blood cells per fetus unfractionated by phenotype, or 1-5×10⁵ CD34⁺ Lin⁻ human bone marrow cells per fetus, 1.1×10⁶ CD34⁺ Lin⁻ human cord blood cells per fetus or 1.1×10⁶ CD34⁻, Lin⁻ human cord blood cells per fetus by any suitable method, for example, intraperitoneally using a 25 gauge needle by the general technique described in Flake et al. Transplantation of fetal hematopoietic stem cells in utero: the creation of hematopoietic chimeras Science, Vol. 233, p. 766 (1986), which permits the injection of the fetus under direct visualization in an amniotic bubble through a midline laparatomy incision. Following injection of the cells, the myometrium is closed in a double layer and the pregnancy is allowed to proceed.

Human cord blood (CB) cells or human bone marrow cells may be obtained, for example, according to standard procedures upon obtaining consent. Mononuclear cells can be depleted of Lin⁺ cells using mouse anti-hCD3, hCD4, hCD8, hCD11b, hCD19, hCD20, hCD56, and human glycophorin A (hGPA) monoclonal antibodies (BD Immunocytometry, San Jose, Calif.). Samples may be enriched for hCD34+ cells (or alternatively depleted of hCD34+ cells) by using anti-hCD34 microbeads (Miltenyi Biotec Inc., Auburn, Calif.).

Previous studies in sheep show that such transplants result in significant multi-lineage human hematopoietic activity into all blood elements by about 1 month post-transplant and in significant numbers of human hepatocytes at birth (about 3 months post-transplant; 5-40% of total cellularity, depending on the phenotype and dosage of the replacement cells). U.S. Pub No. 20020100065. Further, chimeric livers resulting from such transplantations include not only human hepatocytes that retain functional properties of normal hepatocytes, but also human endothelial and biliary duct cells, and secrete human albumin into the circulation. Almeida-Porada, Formation of human hepatocytes by human hematopoietic stem cells in sheep, Blood, (2004) 104(8) 2582-2590, which is incorporated by reference herein in its entirety. The human cells persist on a long term basis.

The chimeric solid organ or tissue developed within the fetal mammal (i.e., the donor mammal) that bears the broadly-active expression of the suicide gene (such as the chimeric liver of the example) is transplanted to a second non-human mammal (i.e., the host) that does not bear expression of the same suicide gene, where it is supported in a living state. The chimeric donor organ or tissue may be transplanted to the non-human mammal host while the donor mammal is still at a fetal stage or at any stage following birth of the donor mammal. Following transplantation, administration of the prodrug to the host selectively kills the non-human cells of the chimeric organ or tissue that originate from the non-human donor mammal without substantially harming the human cells of the chimeric organ or tissue and without substantially harming the cells of the host, thereby promoting a more complete cellularization of the transplanted solid organ or tissue with the human cells versus the donor mammal cells. In this manner, a more cellularly human organ or tissue can be obtained, such as an at least substantially entirely cellularly human organ or tissue. Such an organ or tissue may, for example, be further transplanted to a human patient in need thereof.

According to one embodiment of the present invention, an organ or tissue of interest, such as a the liver, that has been engrafted with replacement cells to form a chimeric organ or tissue, can be later transplanted to a host mammal (not expressing the suicide transgene) where at least some of the donor mammal cells of the chimeric organ or tissue are selectively deleted (or growth-impaired) thereby allowing the replacement cells (such as human replacement cells) that are present in the chimeric organ or tissue to selectively populate the organ or tissue.

Multiple Animal Transplant Procedures

In one embodiment of the invention, an individual non-human mammal serves as both a donor of a solid organ or tissue or part thereof to another non-human mammal and as a host for a solid organ or tissue or part thereof from a human or non-human mammal. The invention further provides related embodiments in which at least two non-human mammals are provided and of the at least two non-human mammals, at least two serve as both a donor animal and a host animal. Such embodiments increase the efficiency of producing cellularly reconstituted organs and tissues. For example, one embodiment includes the steps of: providing a first non-human mammal that inducibly or constitutively expresses a first suicide gene and a second non-human mammal that inducibly or constitutively expresses a second suicide gene, wherein the set of conditions required to kill cells are different for the first and second suicide genes; transplanting an organ or tissue or part thereof from the first non-human mammal to the second non-human mammal and transplanting an organ or tissue or part thereof from the second animal to the first animal, wherein the same organs or tissues or part(s) thereof are transplanted between the animals (reciprocal symmetrical-type transplant) and/or different organs or tissues or part(s) thereof are transplanted between the animals (reciprocal asymmetrical-type transplant). The transplanted organs or tissues are supported in a living state by their respective hosts. In this manner, for each of the animals, the cells of the transplanted organs or tissues hosted therein can be selectively killed without killing the host cells by providing the set of conditions that kill cells of the respective donor animal cells to the respective host. The same or different promoters or promoter types may be used to drive expression of the first and second suicide genes. The promoters may be broad activity promoters, such as a universal promoter, and/or cell-type or tissue-type specific promoters. An inducible growth-impairing gene that impairs replication and/or health of cells or otherwise selectively disadvantages cells without killing them in response to a set of conditions may also be used instead of one or more suicide genes.

In accordance with the facilitated cellular reconstitution aspects of the invention, replacement cells, such as human replacement cells, may be introduced into the donor organs or tissues by any method, such as before explantation from the respective donors, for example, by introducing stem cells or progenitor cells into a donor animal during its fetal or perinatal stage and/or after explantation from the donor by directly or indirectly introducing replacement cells into a donor organ, before and/or after transplantation to the host. The replacement cells that are introduced into the donor organ or tissue may be the same or different for the two animals and may be from the same source or different sources. In this manner the selective killing or growth impairment of the endogenous cells of a donor organ or tissue within its respective host promotes the selective reconstitution of the organ or tissue by the replacement cells. After sufficient cellular reconstitution of the organ or tissue by the replacement cells, the reconstituted organ or tissue may be explanted from the host. Any contaminating host cells that may be present in the reconstituted organ or tissue may be killed or impaired by then providing the conditions that kill or impair the respective host cells.

In cases in which more than two non-human mammals are employed and individuals act as both donors and hosts, organ or tissue transplants between the individuals may be reciprocal (where two of the individuals exchange organs or tissues or parts thereof with each other) and/or non-reciprocal (transplants are not received from the same individuals to which transplants are given).

Selective Deletion of Donor Cells and Host Cells

The invention provides embodiments in which cells of the donor organ or tissue are conditionally and selectively killable (deletable) versus the host's cells and the replacement cells, in response to a set of one or more conditions. In this manner, the invention provides that at least some of the donor cells of the donor organ or tissue can be deleted in order to facilitate an at least partial reconstitution by replacement cells.

The invention also provides embodiments in which the host cells are deletable, in response to a second set of conditions that is different than the first set of conditions, so that host cells that may have infiltrated a reconstituted organ or tissue can be deleted therefrom. The types of host cells that may migrate into and be present in a reconstituted donor organ or tissue hosted according to the invention may include, for example, fibroblasts, lymphocytes and/or other immune cells, vascular endothelial cells, and/or host-derived organ or tissue-type specific cells corresponding to the human donor organ or tissue type.

Various types of suicide gene strategies can be employed including, but not limited to, the following cases:

Protoxin-to-toxin converting enzyme suicide genes. Examples of suitable converting enzyme suicide genes include, but are not limited to, thymidine kinase (either wild-type or comprising a mutation), cytosine deaminase, carboxylesterase, carboxypeptidase, deoxycytidine kinase, nitroreductase, guanosine xanthin phosphoribosyltransferase, purine nucleoside phosphorylase, and thymidine phosphorylase. In the absence of the protoxin (prodrug), expression of the suicide gene produces no or little adverse effects on normal cellular metabolism. The product of a converting enzyme type suicide gene acts on a suitable prodrug, converting it into a toxin. In the absence of the suicide gene product, the prodrug is relatively innocuous. Suitable prodrugs for thymidine kinase include ganciclovir, 6-methoxypurine arabinoside, and (E)-5-(2-bromovinyl)-2′deoxyuridine. A suitable prodrug for cytosine deaminase is 5-fluorocytosine. A suitable prodrug for carboxylesterase is irinotecan. A suitable prodrug for carboxypeptidase is 4-([2-chloroethyl][2-mesyloethyl]amino)benzyol-L-glutamic acid. Suitable prodrugs for deoxycytidine kinase include 4-ipomeanol cytosine arabinoside and fludarabine. Suitable prodrugs for guanosine-xanthin phosphoribosyl transferase include 6-thioxanthine and 6-thioguanine. A suitable prodrug for nitroreductase is 5-aziridin-2,4-dinitrobenzamidine. A suitable prodrug for purine nucleoside phosphorylase is 6-methylpurine deoxyribonucleoside. Suitable prodrugs for thymidine phosphorylase include 5′-deoxy-5-fluorouridine and 1-(tetrahydrofuryl)-5-fluorouracil.

Cell death inducing suicide genes. Other sorts of suicide genes that can be used according to the invention include those whose gene product, itself, causes or induces cell death. Expression of such a suicide gene and hence cell death can be made conditional by placing expression of the suicide gene under the control of an inducible promoter. One type of cell death inducing suicide gene encodes a protein toxin, such as a diphtheria toxin, that kills cells in which it is expressed. Another type of cell death inducing suicide gene encodes an enzyme that acts on cellular substrates to cause or trigger cell death. For example, suitable cell death causing enzyme genes include those encoding cytotoxic proteases such as members of the ICE/CED-3 family of cysteine proteases and caspases, such as Caspase 8h or Caspase 8i (disclosed in U.S. Pat. No. 6,172,190, which is incorporated by reference herein in its entirety).

Signaling-activated suicide gene mechanisms. Transgenic animals engineered so that contacting cells with a dimerizing agent (or clustering agent generally) activates a signaling pathway causing cell death can also be employed for the present invention. For example, the art provides transgenic animals in which contacting cells with rapamycin or rapalog triggers apoptosis by clustering expressed transgenic fusion proteins that contain intracellular domains of apoptosis mediator molecules, such as the Fas receptor or TNF-R1. Suitable signaling mechanisms are provided, for example, by U.S. Pat. No. 6,649,595, U.S. Pat. No. 6,187,757 and U.S. Pub. No. 20030206891 (application Ser. No. 10/341,967), each of which is incorporated by reference herein in its entirety. In another example, transgenic animals expressing proteins that contain intracellular domains of apoptosis mediator molecules, such as the Fas receptor or TNF-R1 and preselected extracellular epitopes can be used. Divalent or multivalent antibodies recognizing the preselected extracellular epitopes can be contacted with cells expressing these proteins to proximalize (cluster) their intracellular domains and thereby induce apoptosis of the cells.

Growth and replication-impairing gene expression. As described by Rhim, expression of the protease, urokinase-type plasminogen activator (uPA) in hepatocytes (under control of the Albumin promoter) causes replicative impairment and confers a selective populative disadvantage to transgenic hepatocytic cells versus normal xenogeneic hepatocytes introduced into the transgenic liver. In general, any sort of method or system that impairs the replication/growth of the non-human mammal donor cells of interest in comparison to the replacement cells may be used according to the invention.

Negative selection markers generally. In general, any sort of negative selection marker or system that allows or enables the selective killing of non-human mammal cells of interest can be used according to the invention. For example, where a donor organ or tissue either naturally or as a result of genetic modification generally expresses a cell surface epitope that is not expressed by the host or replacement cells, cytotoxic agents can be preferentially targeted to the donor cells (versus the host and replacement cells) using antibodies or other binding proteins that specifically bind the epitope. One or more cytotoxic agents can, for example, be linked directly to such an antibody or binding protein or an immunoliposome displaying the antibody or binding protein and containing the cytotoxic agent(s) can be used to shuttle the agent(s) to the target cells.

Resistance Gene Expression in Host and/or Replacement Cells

Host and replacement cells having resistance to a chemical selection agent, for example, as a result of genetically modifying the hosts and/or cells to express resistance genes, can be used to obtain selective deletion of donor organ or tissue cells, for example, by administering the selection agent to the transgenic host while it is supporting the donor organ or tissue.

Expression of the aminoglycoside 3-phosphotransferase gene product (APH, neo^{R}, kan^{R}) can be used to confer resistant to G418 (Geneticin®). G418 concentrations in the range of, e.g., 10-1,000 mg/l such as 400 mg/l or 10-1000 mg/kg (drug weight to host weight), such as 400 mg/kg can be used.

Expression of bsdgene product (from Aspergillus terreus) can be used to confer resistance to Blastocydin S. Blastocydin S concentrations in the range of, e.g., 1-100 mg/l such as 10 mg/l effective or 1-100 mg/kg, such as 10 mg/kg, can be used.

Expression of the sh ble gene product (from Streptoalloteichus hindustanus) can be used to confer resistance to Zeocin®, a member of bleomycin family. Zeocin concentrations in the range of, e.g., 400-1000 mg/l or 400-1000 mg/kg can be used.

Expression of the hph gene product can be used to confer resistance to Hygromycin B. Hygromycin B concentrations in the range of, e.g., 50-1000 mg/l or 50-1,000 mg/kg can be used.

Blastocydin S, Zeocin and Hygromycin B are each effective as selection agents for a number of different types of animal cells and each is known in the art to be effective in mammalian cells. The suggested working concentrations are based on use for mammalian cells. Effective concentrations and doses for any animal or cell type can be determined by routine experimentation. Codon-optimized versions of these genes for mammalian or other expression are known in the art and are commercially available.

Streptozotocin preferentially kills pancreatic islet cells and induces diabetes. Transgenic non-human mammal hosts over-expressing the protein metallothionein in at least pancreatic [beta]-cells, for example under control of a broad-activity promoter or the insulin promoter, are resistant to streptozotocin. Chen et al. Overexpression of metallothionein in pancreatic [beta]-cells reduces streptozotocin-induced DNA damage and diabetes, Diabetes 50:2040-2046, 2001. Transgenic streptozotocin-resistant hosts can also be provided, for example, by expressing streptozotocin-binding moieties such as peptides and/or aptamers that sequester streptozotocin from its DNA target in the host cells. Suitable dosage ranges of streptozotocin for destroying pancreatic islet cells include, e.g., 1-100 mg/kg, such as 30-60 mg/kg.

Promoters. Broad-activity promoters (with respect to cell types) for driving gene expression, such as suicide gene expression, are those active in many cell types, at least substantially in all cell types (a universal promoter), or at least active in at least substantially all of the cell types that are relevant to a subject host, donor organ or tissue, or provider cells from a replacement cell provider, as used in embodiments of the invention. Broad activity promoters can be constitutive or inducible. The term “promoter” as used herein should be construed broadly, for example, as including promoters and enhancers and combinations thereof.

Suitable broad-activity constitutive promoters include, but are not limited to, the MoMLV LTR, RSV LTR, Friend MuLv LTR, adenovirus promoter, neomycin phosphotransferase promoter/enhancer, late parvovirus promoter, Herpes TK promoter, SV40 promoter, metallothionen IIa gene enhancer/promoter, cytomegalovirus immediate early promoter, and cytomegalovirus immediate late promoter. Suitable broad-activity inducible promoters or inducible expression systems can include, but are not limited to, an inducible metallothionein gene promoter, a tetracycline repressor and/or activator based inducible expression system (e.g., as provided by U.S. Pat. Nos. 6,252,136; 6,136,954; 5,912,411; and 5,589,362, each incorporated by reference herein in its entirety); a lac operon based inducible expression system (e.g., as provided by U.S. Patent Appin. Publication 20040171824 (application Ser. No. 10/469,881, which is incorporated by reference herein in its entirety); or an ecdysone inducible expression system ( e.g., as provided by U.S. Publication 20020187972 (application Ser. No. 09/949,278), which is incorporated by reference herein in its entirety). The use of broad-activity regulatory elements, such as a universal promoter, to drive suicide gene expression in donor organs or tissue simplifies the selective deletion of the donor cells to facilitate reconstitution by replacement cells. Similarly, the use of a broad activity promoter to drive expression of a positive selection marker such as a resistance gene in host cells and replacement cells also simplifies the selective deletion of donor organ or tissue cells where the donor organ or tissue cells are not resistant. The use of broad-activity promoters also simplifies deletion of contaminating host cells that may be present in a reconstituted organ or tissue. However, the invention also provides that one or a combination of tissue or cell-type specific promoters and regulatory elements generally can also be used. As referred to herein, tissue-specific and cell-type-specific transcriptional regulatory elements, such as promoters and enhancers and combinations thereof, also include tissue-preferred and cell-type preferred transcriptional regulatory elements. Tissue-specific promoters may also be used in conjunction with broad activity promoters to obtain sequential cellular replacements, as described hereinabove.

Numerous suitable tissue-specific and cell-type specific transcriptional regulatory elements are known in the art. The identification and characterization of further tissue-specific and cell-type specific elements, for a given tissue or generally, is a matter of routine research and a common occurrence in the art. Accordingly, the following examples are provided for illustration and in no way limit the invention to only those elements recited herein.

Hepatocyte and/or hepatocytic cell-specific expression can be provided, e.g., by the albumin promoter and, for fetal-specific liver expression, by the alpha-fetoprotein promoter.

Muscle specific expression can be provided, e.g., by the myosin light chain-2promoter, alpha actin promoter, troponin 1 promoter, Na⁺/Ca²⁺ exchanger promoter, dystrophin promoter, creatine kinase promoter, alpha7 integrin promoter, troponin C promoter-enhancer, alpha B-crystallin/small heat shock protein promoter. Cardiac muscle specific expression can be provided, e.g., by the alpha-myosin heavy chain promoter and the atrial natriuretic factor (ANF) promoter.

Endothelial cell-specific expression can be provided, e.g., by gene promoters for the fms-like tyrosine kinase-1 (Flt-1), intercellular adhesion molecule-2 (ICAM-2), von Willebrand factor (vWF), and Vascular Endothelial Growth Factor Receptor-2 (Flk-1). The Flt-1 promoter reportedly directs expression in all vascular beds except those of the liver.

Lung specific promoters include those for the various lung surfactant proteins, such as the surfactant protein B promoter.

Expression in lymphocytes and/or their progenitors can be provided, e.g., by the jak3 (Janus kinase 3) gene promoter or the LCK gene promoter, which normally drives expression of a lymphocyte-specific protein tyrosine kinase.

Kidney-specific expression can be provided, e.g., by the Ksp-cadherin gene promoter or the human PTH/PTHrP receptor gene kidney-specific promoter.

Epidermal cell specific expression can be provided, e.g., by the human epidermal type 1 transglutaminase (TGase I) gene promoter (U.S. Pat. No. 5,643,746, incorporated by reference herein in its entirety).

Adipose specific expression can be provided, e.g., by the fat-specific promoter/enhancer of the fatty acid-binding protein gene, alpha-P2.

Pancreas-specific expression can be provided, e.g., by the endocrine pancreas-specific insulin promoter (plus or minus the first intron), pancreas alpha-amylase promoters, the pancreas-specific duodenum homeobox 1 (PDX-1) promoter; and the exocrine pancreas-specific promoter of the elastase I gene (Hall et al., J., Biotechnology (1993) 11: 376-379).

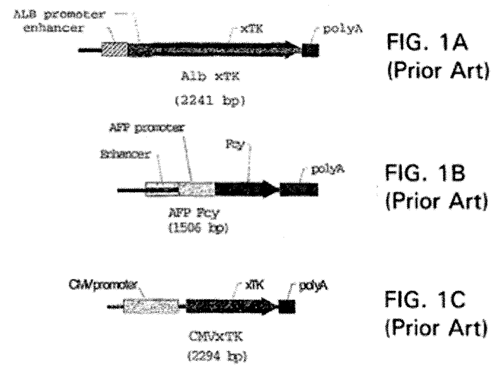

Numerous suicide gene expression constructs and methods including those already described in the art can be employed for providing non-human mammals having conditionally deletable (killable) cells, for use according to the invention. For example, the following sequences and the methods provided by International Publication WO 2004/027029 A2 (International Application PCT/US2003/029251) can be used. SEQ ID NO: 1 provides the sequence of the porcine albumin promoter. SEQ ID NOS. 2-5 provides transgene constructs for the production of transgenic non-human mammals expressing a preselected suicide gene. Specifically, SEQ ID NO: 2 provides a transgene construct including a mutant form of the herpes thymidine kinase suicide gene (xTK) under control of the liver specific porcine Albumin promoter and a poly-A addition signal sequence for the transcript. SEQ ID NO: 3 provides a transgene construct including the suicide gene cytosine deaminase (fCY) under control of the fetal liver-specific alpha-fetoprotein promoter, and a poly-A addition signal sequence for the transcript. SEQ ID NO: 4 provides a transgene construct including a mutant form of the herpes thymidine kinase suicide gene (xTK) under control of a broad-activity, constitutive cytomegalovirus (CMV) promoter, and a poly-A addition signal sequence for the transcript. SEQ ID NO: 5 provides a transgene construct including the suicide gene cytosine deaminase (fCY) under control of a broad-activity, constitutive cytomegalovirus (CMV) promoter, and a poly-A addition signal sequence for the transcript. For illustration, FIG. 1 shows the arrangement of elements of the transgene constructs of SEQ ID NO: 2 (Alb xTK), SEQ ID NO: 3 (AFP Fcy), and SEQ ID NO: 4 (CMV xTK).

xTK is a mutated version of a Herpes simplex virus (HSV) thymidine kinase gene characterized by the substitution of adenosine for cytosine at base positions 130 and 180. The nucleotide substitutions result in a codon changes from leucine to methionine and prevent the phenomenon of male sterility that reportedly can occur with the unmodified form. These mutations do not substantially impair enzymatic activity. The constructs of SEQ ID NOS: 2-5 may also include the coding sequence for a form of green fluorescent protein (GFP) under control of a universal promoter. GFP expression allows host cells to be identified visually and be easily distinguished from human cells. However, GFP expression is an optional feature that is not necessary for implementation of the invention.

Those skilled in the art will recognize that there are several methods for producing transgenic animals and cell lines and that any suitable method can be employed.

Example—Production of Transgenic Pigs Expressing a Suicide Gene

This example illustrates the production of transgenic pigs containing a suicide gene expression construct using a somatic cell nuclear transfer technique, as known in the art. Briefly, fibroblasts from 35-day-old fetal pigs are cultured and then transfected with a suicide transgene construct (e.g., either a mutated thymidine kinase or cytosine deaminase construct) using electroporation or any suitable technique. Coichicine is added to arrest the transfected fibroblasts at the G2/M phase. Swine oocytes are isolated and enucleated. For each of several or many enucleated oocytes, a transfected fibroblast is inserted in the perivitelline space using a micromanipulator and electrofusion is then employed to effectively transfer the donor fibroblast nucleus into the enucleated oocyte. Electrofusion and activation can be performed simultaneously or activation can be performed after the electrofusion step. In an alternative method of transfer, the somatic donor nucleus can be microinjected into the enucleated oocyte, followed by activation. In either case, following activation, the reconstructed embryos are implanted into surrogate sows at estrus. The litters can be monitored by ultrasound. At term, the transgenic pigs may be delivered by Caesarean section, if desired.

The presence of the suicide transgene construct(s) in the pigs can be assessed using PCR. Expression of the transgene can be evaluated by Western blotting. The transgenic pigs can be bred once they reach sexual maturity. Pigs homozygous for the suicide gene construct can be obtained by breeding, if desired. Further transgenic pigs can be obtained by breeding and/or cloning.

Suitable dosages for the administration of prodrugs and/or inducers of transcription and/or multimerizing/dimerizing agents can be empirically determined as a matter of routine experimentation. Suitable and optimal dosages may vary with different types of hosts and expression constructs. For example, such agents may be administered to a non-human animal host at a dose of 1-1,000 mg/kg, 1-100 mg/kg, or 5-50 mg/kg. For pigs expressing thymidine kinase, an effective dose of ganciclovir can, e.g., be 1-1,000 mg/kg, 1-100 mg/kg, 5-50 mg/kg, or about 25 mg/kg. For ex vivo killing of non-human host cells present in explanted humanized donor organs or tissues, a concentration range of 1-1000 mg/l, 1-100 mg/l, 5-50 mg/l or 20-50 mg/l can, for example, be used. For deleting pig host cells expressing thymidine kinase from explanted humanized donor organs or tissues, ganciclovir concentrations of 2-1000 mg/l, such as about 100 mg/l can, for example, be used. For ex vivo thymidine kinase-based negative selection using the prodrug 5-BrdU (5-Bromo-2′-deoxyuridine), a concentration range of 1-1000 mg/l, 1-100 mg/l, or 25-30 mg/l, can, for example, be used.

In embodiments in which the process of selectively killing host cells is begun or initiated while the reconstituted donor organ or tissue still remains in the host, the agent(s) necessary for beginning or initiating such killing can be administered to the host by, for example, intravenous injection. For example, in embodiments where expression of a converting enzyme type of suicide gene is inducible, such induction can be begun before the reconstituted donor organ(s) or tissue(s) are explanted. The prodrug can then be administered also while the donor organ(s) or tissue(s) remain in the host and/or contacted with the donor organ(s) or tissue(s) after removal from the host.

For ex vivo killing of host cells, the agents necessary for negative selection may be prepared in liquid medial that is contacted with the explanted organ or tissue, for example, by immersion in such medial and/or by perfusion with such media.

Reducing Transfer of Xenoantigens from the Host to the Reconstituted Organ or Tissue

Certain embodiments of the invention are based on the recognition that cell surface antigens of a non-human mammal host can be transferred, by natural mechanisms, to the cell surface of foreign cells that are resident within the host mammal. One embodiment of the invention provides for reducing the expression of at least one xenoantigen in a non-human mammal host in order to reduce or eliminate its transfer to foreign donor cells, organs and/or tissues and/or replacement cells that are resident in and supported by the non-human mammal host. For example, the transfer of major xenoantigens for humans, such as alpha-galactosyl epitopes, i.e., Gal.alpha.(1,3) Gal epitopes, and/or minor xenoantigens for humans can be reduced.

As referred to herein, glycosylphosphatidylinositol-anchored. proteins are proteins bound to the lipid bilayer of a membrane through either a glycosylphosphatidylinositol anchor (GPI-anchor), which is a complex oligoglycan linked to a phosphatidylinositol group, or a GPI-like-anchor, i.e., a similar complex oligoglycan linked to a sphingolipidinositol group, resulting in the attachment of the C-terminus of the protein to the membrane. Certain extracellular carbohydrate epitopes are also directly linked to cell membrane lipids.

Glycosylphosphatidylinositol (GPI) anchored proteins are known to be exchanged between the membranes of living cells in vivo, for example, from erythrocytes to endothelium and vice versa. Medof et al., Cell-surface engineering with GPI-anchored proteins, Cell Surface Eng'g (1996) Vol. 10, pp. 574-586; Kooyman et al. (1995) In vivo-transfer of GPI-linked complement restriction factors from erythrocytes to endothelium Science, Vol. 269, pp. 89-92. GPI-anchored proteins are also known to be exchanged between the membranes of erythrocytes. Sloand et al. (2004) Transfer of glycosylphosphatidylinositol-anchored proteins to deficient cells after erythrocyte transfusion in paroxysmal nocturnal hemoglobinuria Blood (12):3782-3788. See also: Babiker et al. (2005) Transfer of functional prostasomal CD59 of metastatic prostatic cancer cell origin protects cells against complement attack Prostate. 62(2):105-114; Dunn et al. (1996) A knock-out model of paroxysmal nocturnal hemoglobinuria: Pig-a(−) hematopoiesis is reconstituted following intercellular transfer of GPI-anchored proteins Proc Natl Acad Sci USA. 93(15):7938-7943; and Anderson et al. (1996) Intercellular transfer of a glycosylphosphatidylinositol (GPI)-linked protein: release and uptake of CD4-GPI from recombinant adeno-associated virus-transduced HeLa cells Proc Natl Acad Sci USA. 93(12):5894-5898. GPI-anchored/associated biomolecules are believed to be transferred between cells via cell-to-cell contact, via microvesicles and/or via microparticles, such as lipoprotein particles. The present invention is not limited by the mechanism of intercellular transfer. Proteins that are loosely embedded in the cell membrane, such as those with short tails embedded in, but not traversing the cell membrane, may also be subject to intercellular transfer by natural mechanisms.

In embodiments of the invention in which donor cells, e.g., in the form of organs or tissues or parts thereof, are to be supported in a living state by a host mammal, at least partially reconstituted with replacement cells and later transplanted or transferred to a recipient mammal, such as a human patient, antigens transferred from the host mammal to the donor material and/or replacement cells can contribute to immunological rejection of the reconstituted organs or tissues by the recipient. Such xenoantigens can, for example, include peptide epitopes of transferred proteins and/or carbohydrate epitopes present on the transferred proteins, such as alpha-galactosyl epitopes and N-glycolyineuraminic acid (NeuGc) epitopes, as well as carbohydrate epitopes directly linked to cell membrane lipids or otherwise linked to the cell membrane.

Alpha-Galactosyl Epitopes

In the case where the host mammal is of the type that produces alpha-galactosyl (Gal.alpha.(1,3)Gal) epitope modified proteins and/or lipids, such as an ungulate or rodent, and the hosted cells comprise alpha-galactosyl epitope negative cells (i.e., cells not producing alpha-galactosyl epitopes), such as human cells, the invention provides for reducing or eliminating completely the amount of alpha-galactosyl epitopes transferred to the epitope-negative cells by employing a non-human mammal host modified to reduce or completely eliminate the expression of alpha-galactosyl epitopes on proteins and/or lipids.

Numerous methods for producing genetically modified animals having reduced expression of alpha-galactosyl epitopes are known in the art including: (1) genetic knock-out of the Gal.alpha.(1,3) galactosyl transferase gene (“alpha-galactosyl transferase gene;” GGTA1) by homologous recombination; (2) expression of transgenes encoding other transferases, such as alpha-fucosyltransferase (e.g., human FUT1 and/or FUT2), that compete with alpha-galactosyltranferase for substrate; and (3) expression of transgenes encoding human N-acetylglucosaminyltransferase III which reduces formation of alpha-galactosyl epitopes by inhibiting N-linked sugar branching. The term “expression” as used herein with respect to a carbohydrate epitope xenoantigen relates to the amount of presentation of the epitope in its xenoantigenic state. Accordingly, reducing the expression of a carbohydrate epitope xenoantigen may, for example, be accomplished by reducing or eliminating the activity of one or more enzymes that produce the carbohydrate epitope xenoantigen, by providing enzyme activities that compete with substrate for such carbohydrate xenoantigen-producing enzymes, by enzymatically cleaving the carbohydrate epitope xenoantigen and/or by otherwise modifying the carbohydrate xenoantigen to a less xenoantigenic structure or state.

Genetically-modified non-human mammals with reduced alpha-galactosyl epitope expression and methods for producing them are provided, for example, by the following patents or applications, each of which is incorporated by reference herein in its entirety: U.S. Pat. No. 6,413,769; U.S. Pat. No. 6,331,658; U.S. Pat. 6,166,288; U.S. Pat. No. 5,821,117; U.S. Pat. No. 5,849,991; U.S. Pub. No. 20040268424 (application Ser. No. 10/646,970); U.S. Pub. No. 20030203427 (application Ser. No.10/125,994); U.S. Pub. No. 20030068818 (application Ser. No. 10/105,963); U.S. Pub. No. 20020031494 (application Ser. No.10/254,077); U.S. Pub. No. 20030014770 (application Ser. No.10/098,276) U.S. Pub. No. 20040073963 (application Ser. No.10/362,429); U.S. Pub. No. 20040171155 (application Ser. No.10/762,888); and U.S. Pub. No. 20030131365 (application Ser. No.10/172,459). Mice homozygously deficient for the GGTA1 gene and methods for making the same are, for example, provided by U.S. Pat. No. 5,849,991. Swine homozygously deficient for the GGTA1 gene and methods for making the same are, for example, provided by U.S. Pub. No. 20040268424. SEQ ID NO: 6 provides the mRNA sequence of a porcine alpha(1,3)galactosyl tranferase gene (GGTA1). Sheep and cow mRNA sequences for the GGTA1 gene are provided in GenBank accession nos. NM_{—}001009764 [SEQ ID NO: 7] and NM_{—}177511 [SEQ ID NO: 8], respectively. The mouse mRNA sequence for the GGTA1 gene is provided, for example, in Genbank accession no. NM_{—}010283 [SEQ ID NO: 9].

Isogloboside 3 (iGb3) synthase is another enzyme that, in addition to alpha(1,3)-galactosyltransferase, synthesizes Galα(1,3)Gal motifs. In contrast to alpha(1,3)-galactosyltransferase, iGb3 synthase preferentially modifies glycolipids over glycoprotein substrates. (Keusch et al. (2000) Cloning of Gb3 synthase, the key enzyme in globo-series glycosphingolipid synthesis, predicts a family of alpha 1,4-glycosyltransferases conserved in plants, insects, and mammals J. Bio. Chem. 275:25308-25314.) iGb3 synthase acts on lactosylceramide (LacCer (Gal.beta.1,4 Glc.beta.1 Cer)) to form the glycolipid isogloboid structure iGb3 (Gal.alpha.1,3 Gal.beta.1,4 Glc.beta.1-Cer), initiating the synthesis of the isoglobo-series of glycoshingolipids. Genetically-modified swine having reduced or eliminated expression of iGb3 synthase and methods and sequences for producing the same are provided, e.g., by U.S. Pub. No. 20050155095 (application Ser. No. 10/981,935), which is incorporated by reference herein in its entirety. The mRNA sequence of the rat iGb3 synthase gene has been reported in GenBank accession no. NM_{—}138524 [SEQ ID NO: 10] and that of the mouse gene in GenBank accession no. NM_{—}001009819 [SEQ ID NO: 11].

According to the invention, the expression of a selected enzyme such as alpha-galactosyl transferase (GGTA1), iGB3 synthase or CMP-NeuAc hydroxylase or a protein xenoantigen may be reduced or completely eliminated in a non-human mammal host (or non-human mammal or organ or tissue thereof generally) by post-transcriptional silencing employing dsRNA (RNA interference, RNAi) and/or transcriptional gene silencing employing dsRNA and/or by antisense methods. Double-stranded RNA molecules used for such silencing may be produced within cells of the host from the host genome or from a vector introduced into the cells and/or may be exogenously provided to the cells. Any of the forms of dsRNA that induce post-transcriptional silencing and/or transcriptional gene silencing can be used including, but not limited to, siRNA (e.g., digestion products of an RNAse IlI such as Dicer or similarly sized and configured short dsRNA molecules), short hairpin RNA, and designed microRNA (miRNA). The design and selection of effective molecules and strategies for RNA silencing and antisense regulation of preselected targets is well established in the art. Nucleotide sequences for porcine alpha-galactosyl transferase (GGTA1) are provided, for example, by U.S. Pat. No. 5,849,991, U.S. Pat. No. 5,821,117 and U.S. Pub. No. 20030203427, each of which is incorporated by reference herein in its entirety.

It should further be understood that the reduction and/or elimination of xenoantigens and/or xenoantigen-producing enzymes can be, but is not necessarily, performed prior to the introduction of the human cells into the non-human mammal host. For example, human cells may be introduced into a fetal non-human mammal host to integrate into one or more organs and tissues of the host and following birth of the host, the reduction or elimination of a xenoantigens and/or xenoantigen-producing enzyme may be induced by any method, such as treatment with RNA silencing molecules that silence expression of a xenoantigen or a xenoantigen-producing enzyme. A transgenic host may also be provided in which RNA silencing of a xenonatigen or xenoantigen-producing enzyme can be induced and/or in which the expression of an enzyme that cleaves or interferes with production of a xenoantigen can be induced.

In one embodiment of the invention, the host and/or donor has reduced expression of alpha(1,3)galactosyl transferase-synthesized Gal.alpha.(1,3)Gal epitopes, for example, as a result of a modification and/or of treatment. In another embodiment, the host and/or donor has reduced expression of iGb3 synthase-synthesized Gal.alpha.(1,3)Gal epitopes, for example, as a result of a modification and/or of treatment. In a related embodiment, the host and/or donor has reduced expression of both alpha(1,3)galactosyl transferase-synthesized and iGb3 synthase-synthesized Gal.alpha.(1,3)Gal epitopes.

Alpha-galactosyl epitopes expressed on host cells that could be transferred to cells that do not express such epitopes, as well as alpha-galactosyl epitopes already transferred to cells that do not express alpha-galactosyl epitopes, can also be removed enzymatically, for example, by alpha-galactosidase or endo-beta-galactosidase C. Such enzymes can, for example, be infused intravenously into a host supporting a human donor organ or tissue and/or expressed constitutively or inducibly in a suitable host. Enzymatic removal of alpha-galactosyl epitopes is taught, for example, by U.S. Pat. No. 6,758,865; U.S. Pat. No. 6,491,912; U.S. Pat. No. 6,331,319; U.S. Pat. No. 6,046,379, and Maruyama et al., Xenotransplantation 11(5), pp. 444-51 (2004), each of which is incorporated by reference herein in its entirety.

In embodiments of the invention where human replacement cells are used to at least partially reconstitute a non-human mammal donor organ or tissue for later transplant to a human, it is preferred that the donor organ or tissue is also negative for alpha-galactosyl epitopes and/or other xenoantigens as described herein, especially if the final organ or tissue product is chimeric, i.e., containing not only human replacement cells but also donor animal cells. Where the replacement cells are non-human and the intended recipient is human, it is also preferred that the replacement cells are negative for the xenoantigens. In a related embodiment, the replacement cells or the provider thereof has reduced expression of alpha(1,3)galactosyl transferase-synthesized and/or iGb3 synthase-synthesized Gal.alpha.(1,3)Gal epitopes, for example, as the result of a modification and/or treatment.

N-Glycolyineuraminic Acid (NeuGc) Epitopes

N-acetyineuraminic acid (NeuAc) and N-glycolyineuraminic acid (NeuGc) are abundant forms of sialic acid that are found as cell surface carbohydrate modifications to proteins and lipids. NeuGc is present in most animals with the notable exception of humans and chickens. Thus, NeuGc is a xenoantigen with respect to the human immune system. NeuGc is synthesized in vivo from N-acetyineuraminic acid (NeuAc) by the addition of a single hydroxyl group by cytidine monophospho-N-acetyineuraminic acid hydroxylase (CMP-NeuAc hydroxylase). According to the present invention, non-human mammals, or organs or tissues thereof, with reduced or completely eliminated expression of NeuGc epitopes can be produced by any suitable method such as (i.) genetic knockout of the CMP-NeuAc hydroxylase gene by homologous recombination, (ii.) post-transcriptional RNA silencing, dsRNA-mediated gene silencing of transcription, and/or antisense techniques against the CMP-NeuAc hydroxylase gene, and/or (iii.) enzymatic removal of NeuGc epitopes using a suitable enzyme such as neuraminidase. Neuraminidase removes both NeuGc and NeuAc cell surface epitopes. If desired, the NeuAc epitope can be regenerated by further treatment with sialyltransferase, using cytidine monophospho-N-acetyineuraminic acid (CMP-NeuAc) as a substrate. The production of non-human mammals genetically modified to eliminate CMP-NeuAc hydroxylase gene expression and nucleotide sequences required therefor, as well as methods for enzymatic removal of NeuGC epitopes are provided by U.S. Pub. Nos. 20030165480 (application Ser. No. 10/135,919) and 20050223418 (application Ser. No 10/863,116), each of which is incorporated by reference herein in its entirety. See also International Pub. No. WO 2004/108904. SEQ ID. NO. 12 is a partial mRNA coding sequence of the porcine CMP-NeuAc hydroxylase gene, derived from U.S. Pub. No. 20030165480. The mRNA sequence of the major and minor alternatively spliced forms of the mouse CMP-NeuAc hydroxylase gene are provided by Genbank accession nos. AB061276 [SEQ ID NO: 13] and AB061277 [SEQ ID NO: 14], respectively.

Animals or organs and tissues thereof that are characterized by reductions in both alpha-galactosyl epitopes and NeuGc epitopes may also be used according to the invention. In one embodiment of the invention, a double gene knock-out, non-human mammal, for example an ungulate, that is homozygously negative for both alpha-galactosyltransferase and CMP-NeuAc hydroxylase is used as a non-human mammal host (and/or donor and/or provider of replacement cells). Heterozygous knockouts are also within the scope of the invention. In another embodiment, the non-human mammal host (and/or donor and/or provider of replacement cells) has either or both of the alpha-galactosyltransferase and CMP-NeuAc hydroxylase genes knocked-out (homozygously), and includes a transgene directing the expression of at least one tolerance-promoting biomolecule. As an alternative to simple gene deletions, genetic knock-outs used according to the invention may also be conditionally obtained, optionally in a tissue-specific manner, using, for example, inducible recombinase expression methods and systems, such as the CRE-LOX system, for gene deletion, as known in the art.

In addition to the intercellular transfer of xenoantigens from host cells to foreign donor cells and/or foreign replacement cells, host xenoantigens can, at least in some instances, also be present in a hosted human organ or tissue in the form of living or dead host cells and/or fragments, such as cell membrane fragments, thereof. Accordingly, one embodiment of the invention provides a method for causing hosted human cell-reconstituted organs or tissues to be better tolerated upon transplantation to a human recipient by using a non-human mammal host that is genetically modified to decrease or completely eliminate the expression of at least one xenoantigen that is not intercellularly transferable from host to donor cells or replacement cells, such as a xenoantigenic host transmembrane protein that is not intercellularly transferable. Examples of xenoantigenic transmembrane proteins, with respect to a human recipient immune system, include non-human, transmembrane MHC class I and MHC class II molecules.

Transfer of Tolerance Promoting Biomolecules from Host to Donor Organ or Tissue

Another aspect of the invention provides a non-human host mammal modified to express or increase its expression of at least one transferable “tolerance promoting” biomolecule that when transferred to donor cells and/or replacement cells (human and/or non-human) of a reconstituted donor organ or tissue, improves the tolerability of the reconstituted product organ or tissue to the immune system of a preselected type of intended recipient, such as a human.

Methods for expressing selected GPI-anchored proteins are well established. For example, several complement inhibiting factors such as human or non-human forms of DAF (decay accelerating factor; CD55), MIRL (membrane inhibitor of reactive lysis, CD59) and MCP (membrane cofactor protein, CD46) occur in GPI-anchored forms. These complement inhibitors are found, for example, on red blood cells and the endothelium, which is a critical site for immunological tolerance or rejection. In one embodiment of the invention, transgenic non-human host mammals expressing or having increased expression of (versus normal endogenous expression) a human or non-human form of at least one of these GPI-anchored complement inhibitors is employed as an animal host of a donor organ or tissue that is or will be reconstituted with replacement cells. A related embodiment provides expression of at least one tolerance-promoting biomolecule, such as a protein, that is not naturally present, or increased expression of a tolerance-promoting biomolecule that is naturally present, by a non-human mammal host, such as but not limited to at least one of the listed complement inhibitors, whereby said expression results in transfer or increased transfer of the tolerance promoting biomolecule(s) to at least some of the foreign cells (the donor cells and/or the replacement cells) resident in the host mammal.

Further, methods for expressing the extracellular domain, or one or more selected portions thereof, of a selected protein that is not regularly expressed in a GPI-anchored form, as a GPI-anchored protein or a GPI-anchored fusion protein are well established in the art and can be used according to the invention to create transgenic non-human mammal hosts in which selected tolerance-promoting transgene products are transferable to foreign cells resident in the host, such as donor cells and/or replacement cells.

GPI-anchored proteins, like other membrane-associated proteins, are modified by the addition of carbohydrate moieties. For example, human CD59 has a single N-glycosylation site and a number of potential O-glycosylation sites. Rudd et al. The glycosylation of the complement regulatory protein, human erythrocytes CD59. (1997) J. Biol. Chem., 272, 7229-7244. Accordingly, one embodiment of the invention provides a non-human host mammal that is genetically modified to express a transferable tolerance promoting biomolecule, such as hCD59, and which also has reduced expression of at least one carbohydrate xenoantigen, such as alpha-galactosyl or NeuGc epitopes, e.g., as the result of a modification such as a genetic modification as described herein. Such a host can be used, in any manner described, to support human organs, tissues and/or cells in a living state. Advantageously, the use of such a host prevents the modification of tolerance-promoting biomolecules, such as tolerance-promoting proteins, with undesirable carbohydrate xenoantigens and thus, prevents their transfer to the hosted human organs,tissues and/or cells while improving the tolerance promoting effect of the transferred tolerance-promoting biomolecule(s).

Another embodiment provides a method including the steps of hosting a reconstituted chimeric or at least substantially human organ in a living state in a non-human mammal host that is genetically modified to express at least one transferable tolerance-promoting biomolecule that is subject to in vivo glycosylation and thereafter enzymatically treating the organ or tissue product, for example, after explantation, to remove carbohydrate xenoantigens, such as those transferred from the host to the organs or tissue product, for example, those that may even be attached to the tolerance-promoting biomolecule(s).

The following examples illustrate various tolerance-promoting biomolecules for expression in transgenic mammal hosts according to the invention and/or provide such hosts.

(i.) U.S. Pat. No. 6,825,395 and U.S. Pub. No. 20030165480, each incorporated by reference herein in its entirety, provide transgenic non-human mammals expressing hDAF.

(ii.) U.S. Pat. No. 6,639,122, incorporated by reference herein in its entirety, provides transgenic swine expressing HLA-D.

(iii.) Transgenic mammals expressing membrane-tethered fusion protein forms of one or both of the anticoagulants human tissue factor pathway inhibitor and hirudin can be used. See Chen et al., Complete inhibition of acute humoral rejection using regulated expression of membrane-tethered anticoagulants on xenograft endothelium. Am J Transplant. December 2004; 4(12):1958-63 and U.S. Pat. No. 6,423,316, each of which is incorporated by reference herein in its entirety.

(iv.) Transgenic mammals expressing human HLA-G to protect from lysis by human NK cells can be used. Human natural killer (NK) cells, which can directly lyse porcine endothelial cells, play an important role in xenotransplantation. HLA-G is a nonclassical major histocompatibility complex (MHC) class I molecule that has been implicated in protecting susceptible target cells from lysis by NK cells. Wang et al., A study of HLA-G1 protection of porcine endothelial cells against human NK cell cytotoxicity. Transplant Proc. October 2004; 36(8): 2473-4.

(v.) Transgenic mammals expressing cell surface human Fas ligand, which induces apoptosis of Fas Receptor bearing cells, can be used. Rodriguez-Gago et al., Human anti-porcine gammadelta T-cell xenoreactivity is inhibited by human FasL (Fas ligand) expression on porcine endothelial cells, Transplantation. Aug. 15, 2001; 72(3):503-9. Since human cells of a human donor organ or tissue that has been supported in a non-human mammal host can appear non-human to the immune system of a human recipient due to transferred host antigens, the invention also provides that the host can express human FasL that can be transferred to the human donor organ or tissues in order to limit immune rejection against the human cells upon further transplantation to a human being. FasL naturally occurs as a transmembrane protein. According to the invention, the extracellular domain of human FasL, such as amino acids Leu 107 to Leu 281, can also be expressed as a GPI-anchored protein or fusion protein, in a monomeric or multimeric form, either constitutively or inducibly, in a transgenic non-human host mammal.

As described above, in addition to the intercellular transfer of xenoantigens from host cells to donor cells and/or replacement cells, host xenoantigens can, at least in some instances, also be present in a reconstituted organ or tissue product in the form of living or dead host cells and/or fragments, such as cell membrane fragments, thereof. Accordingly, one embodiment of the invention provides a method for causing reconstituted organs and tissues to be better tolerated upon transplantation to a human recipient by using a non-human mammal host that is genetically modified to express or increase the expression of at least one tolerance-promoting biomolecule, which is or is not intercellularly transferable from host to donor cells. In this manner, the tolerance-promoting biomolecule(s) at least partially ameliorates recipient immune reactions to xenoantigens present on the host cells or fragments and thereby reduces the general recruitment of a negative immune response toward the reconstituted organ or tissue in a human recipient. Host cell fragments and/or extracellular matrix may be present in the reconstituted organ or tissue even after host cells therein have been selectively killed if they have not had time to clear and/or have not been actively cleared. Methods for clearing reconstituted human organs and tissues of host antigens and cellular material are provided by further embodiments of the invention described below.

Post-Conditioning Embodiments

A further aspect of the invention provides methods for conditioning reconstituted organs and tissues, such as those containing human replacement cells, that have been supported in a living state in a non-human mammal host to be better tolerated by the immune system of a preselected type of recipient, such as a human being. In one embodiment, the reconstituted organ or tissue is isolated from the mammal host's circulation, for example, by explantation from the host, and is at least partially cleared of xenogeneic (with respect to the preselected type of recipient) cells, xenogeneic cellular material, xenogeneic extracellular material and/or xenogeneic antigens that may be present in the organ or tissue. In a related embodiment, the treated organ or tissue is then transplanted to the preselected type of recipient.

In one embodiment, major and/or minor xenoantigens from the non-human mammal host that are present within the reconstituted donor organ or tissue, for example membrane linked proteins and/or carbohydrate epitopes that were transferred from the host to the engineered organ or tissue product or cellular debris of host cells are, at least in part, passively cleared from the organ or tissue after isolation from the mammal host's circulation as a result of their natural turnover and degradation.

In another embodiment, the removal of xenogeneic material from the reconstituted product organ or tissue is actively facilitated after isolation from the mammal host. In one case according to the invention, the cells of the non-human mammal host are selectively killable over the cells of the donor mammal and/or replacement cells and the reconstituted organ or tissue is subjected to the conditions required to selectively kill unwanted mammal host cells that were resident in the organ or tissue, e.g., by contacting the organ or tissue with the necessary agent(s). The cellular debris that result from this killing process may, for example, be at least partially cleared from the organ or tissue by perfusion of the tissue after isolation from the host. Optionally, the debris may also be filtered out of the perfusate, for example, in the case where the perfusate recirculates through the isolated reconstituted organ or tissue.

In another embodiment, xenogeneic cell surface antigens that may be present within the reconstituted organ or tissue are actively removed or modified enzymatically after isolation from the mammal host by contacting the organ with a medium containing enzymes, for example, by immersion in or perfusion with the medium. For example, the invention provides that carbohydrate xenoantigens that may be present in the reconstituted organ or tissue may be removed by perfusing the organ or tissue with a medium containing an appropriate glycosidase, such as an alpha-galactosidase or endo-beta-galactosidase C (EndoGalC) for removing alpha-galactosyl epitopes and/or neuraminidase for removing NeuGc epitopes. (For alpha-gal, see U.S. Pat. No. 6,758,865; U.S. Pat. No. 6,491,912; U.S. Pat. No. 6,331,319; U.S. Pat. No. 6,046,379 and Maruyama et al. Xenotransplantation. September 2004; 11(5): 444-51; and for NeuGc, see U.S. Publication 20030165480 (application Ser. No. 10/135,919)—each of which is incorporated by reference herein in its entirety.) These particular epitopes may, for example, be present when the non-human mammal used as a host has not been genetically modified to completely eliminate their expression.

Similarly, the invention provides that GPI-anchored major and/or minor xenoantigens (the proteins or xenoantigenic moieties linked to the GPI-anchored proteins) may be at least partly removed by generally removing GPI-linked proteins by contacting the reconstituted organ or tissue (e.g., by immersion or perfusion) with a suitable enzyme such as a phosphatidylinositol-specific phospholipase C (PI-PLC) or phosphatidylinositol-specific phospholipase D (PI-PLD). Suitable phospholipases are provided, for example, by U.S. Pat. No. 6,689,598; U.S. Pat. No. 6,638,747; and U.S. Pat. No. 5,418,147, each of which is incorporated by reference herein in its entirety. Advantageously, GPI-anchored xenoantigens arising from the mammal host are thus cleared, while removed GPI-anchored biomolecules specific to the reconstituted organ or tissue product cells (such as those derived from human replacement cells) will be naturally regenerated.

Another embodiment includes initiating or at least partly performing the cell-death inducing treatment or enzymatic treatments described above while the reconstituted organ or tissue is not yet isolated from the mammal host's circulation. The reconstituted organ or tissue can then be isolated from the mammal host before the effects of the treatment are substantially undone by further contact with the host's circulatory system.

Methods and media for perfusing organs and tissues are well developed in the art. Suitable methods and media are provided, for example, by U.S. Pat. No. 6,699,231; U.S. Pat. No. 6,677,150; U.S. Pat. No 6,680,305; U.S. Pat. No. 6,627,393; U.S. Pat. No. 6,589,223; U.S. Pat. No. 6,506,549; U.S. Pat. No. 6,589,223; U.S. Pat. No. 6,677,150; U.S. Pat. No. 6,589,223; U.S. Pat. No. 6,524,785; U.S. Pat. No. 6,100,082; U.S. 5,965,433; U.S. Pat. No. 5,586,438; U.S. Pat. No. 5,498,427 U.S. Pat. No. 5,599,659; U.S. Pat. No. 6,492,103 and U.S. Pat. No. 5,362,622, each of which is incorporated by reference herein in its entirety.

Extra-Corporeal Support Embodiments

A related method of the invention includes the steps of explanting the reconstituted organ or tissue from the non-human mammal host in which it was supported and thereafter supporting the organ or tissue in a living state in isolation from the non-human mammal host using an extracorporeal support device and/or method, for a period of time. During the period of extracorporeal support, at least some xenogeneic (with respect to the preselected type of recipient) cells, xenogeneic cellular material, xenogeneic extracellular material and/or xenogeneic antigens, from the non-human host mammal and/or undesirably remaining from the non-human donor mammal (e.g., where total replacement by replacement cells is desired), are actively and/or passively removed (cleared) from the organ or tissue in, for example, the same manners described above. In a related embodiment, the treated organ or tissue is transplanted to the preselected type of recipient after the period of extracorporeal support. In one embodiment, the period of extracorporeal support is approximately 1, 2, 3, 4, 5, 6, 7, 10 or 14 days. In another embodiment, the period of extracorporeal support is at least 1, 2, 3, 4, 5, 6, 7, 10 or 14 days.

Any type of extracorporeal device and/or method for the support of living donor organs can be used. Some of these devices are similar to heart-lung machines in that they perfuse the subject organ with a medium providing oxygen and nutrients. This medium may, for example, be based at least in part on blood and/or artificial blood, such as a hemoglobin-based blood substitute or a fluorocarbon based blood substitute. One such device is the Transmedics Portable Organ Preservation System (POPS). Suitable extracorporeal support devices and/or methods include, but are not limited to those described in, U.S. Pub. No. 20040171138; U.S. Pat. No. 6,100,082; U.S. Pat. No. 6,046,046; U.S. Pat. No. 6,677,150; U.S. Pat. Nos. 6,673,594; 6,642,045; U.S. Pat. No. 6,582,953; U.S. Pat. Nos. 6,794,182; 5,326,706; U.S. 5,494,822; U.S. Pat. No. 4,837,390; U.S. Pat. No. 4,186,565; U.S. Pat. No. 4,745,759; and U.S. Pat. No. 5,807,737 each of which is incorporated by reference herein in its entirety.

One extracorporeal support embodiment includes the steps of: explanting the reconstituted organ or tissue from the non-human mammal host, thereafter maintaining it in a living state on extracorporeal support for a period of time, and during at least part of the period of extracorporeal support, selectively killing non-human host cells (and/or donor cells) and/or enzymatically treating the organ or tissue to remove xenoantigens, as described above. A related method further includes the step of: after the period of extracorporeal support, transplanting the reconstituted organ or tissue to a recipient, such as a human recipient.

Another extracorporeal support embodiment includes the steps of: initiating or at least partly performing the selective deletion of host cells from the reconstituted organ or tissue and/or enzymatically treating the organ or tissue to remove xenoantigens, as described above, while the organ or tissue is not yet isolated from the mammal host's circulation; explanting the organ or tissue before the effects of the treatment(s) are substantially undone by further contact with the host circulatory system; and thereafter maintaining the organ in a living state on extracorporeal support. One or more of the treatments described can also be performed or continued during support of the organ or tissue by the extracorporeal support device A related method further includes the step of: after the period of extracorporeal support, transplanting the reconstituted organ or tissue to a recipient, such as a human recipient.

Embryo or Fetus Transfer Embodiments

A further embodiment of the invention employs the transfer of an embryo or fetus rather than transplantation of a donor organ or tissue to a host as earlier described above. A non-human mammal embryo or fetus that is heterozygous or homozygous for a transgene conferring tissue specific expression of a suicide gene or any sort of negative selection marker (trait), or otherwise having tissue-specific expression of a negative selection marker, is produced. Embryos can be readily obtained by in vitro fertilization with parent gametes from parent animals having the desired negative selection trait, such as transgenic parent animals (e.g., with respect to one or more desired suicide transgenes). Fetuses can be obtained from pregnant animals.

In one method, each of the parent animals is homozygous for the transgene, or tissue-specific negative selection trait generally, so that every embryo or fetus obtained is also homozygous for the trait. If one parent is homozygous for the trait and the other is homozygous for not having the trait, all progeny will be heterozygous for the trait. So long as one parent is at least heterozygous for the trait (and the trait is dominant), embryos and fetuses heterozygous for the trait can be produced and selected.

The embryos or fetuses are then transferred to a surrogate mother animal of the same or a different species that is not transgenic for the same tissue-specific suicide gene expression, or not expressing the same tissue-specific negative selection marker(s) as a general matter. The transferred embryo or fetus is supported by the surrogate mother. For example, embryos can be transferred to a pseudopregant female by, for example, methods known in the art. Fetuses, for example, can be surgically transplanted into the womb of an already pregnant surrogate mother animal.

Since the mother lacks the negative selection trait of the fetus (that has been transferred or that develops in the mother from a transferred embryo), cells of the fetal tissue that express the negative selection trait can be conditionally and selectively deleted, versus corresponding tissues of the mother. The surrogate mother also provides compensatory support for metabolic functions that may be impaired in the fetus by selectively killing cells of the organ or tissue of the fetus that expresses the negative selection trait.

Accordingly, replacement cells for the organ or tissue that expresses the negative selection trait can be introduced into the fetus and their engraftment and expansion can be facilitated by providing the fetus with the set of conditions that delete cells expressing the negative selection trait. Where the set of conditions includes at least one chemical agent such as a prodrug, it may be introduced into the fetus indirectly by administration to the mother so long as it is able to cross the placenta. Prodrugs that are not able to cross the placenta can be introduced directly into the fetus, for example, via a catheter. Replacement cells may be introduced into the fetus by infusion from a catheter or injected into the target organ or tissue, before, after and/or during the negative selection treatment. Once the replacement cells have engrafted, further negative selection treatments can be used to facilitate the selective reconstitution of the organ or tissue with the replacement cells.

An advantage of this embodiment, in contrast to the invention of Beschorner, is that here fetuses homozygous for the negative selection trait can be used.

In one variation of the embodiment, the replacement cells include or are human cells. In another variation, they include non-human cells. In another variation, the mother and the embryos or fetuses are from the same species, for example, ungulates of the same species, for example, both pigs.

In another variation of the embodiment, the at least partially reconstituted organ or tissue is allowed to grow within the fetal individual after it has been born. Still another variation includes a further step of transplanting the reconstituted tissue or organ from the fetus or from the post-birth individual that was the fetus to another mammal, such as a human. In another variation, one or both of the mother and the fetus is a non-human mammal modified, such as genetically modified, to reduce the expression of xenoantigens, such as those described herein. Where, for example, the replacement cells are also non-human, they may also be modified, such as genetically modified, to reduce their expression of xenoantigens. The reconstituted organ or tissue of this embodiment can also be subjected to any of the post-conditioning treatments, such as enzymatic treatments, and/or extra-corporeal support methods described above.

General Xenoantigen Transfer-Related and Post-Conditioning Treatment Embodiments

The inventor has also recognized that the use of hosts with reduced transferable xenoantigen expression as described herein offers distinct advantages for supporting and/or expanding human cells and/or human cell containing compositions of any sort (e.g., human or chimeric human, non-human organs or tissues, including, e.g., parts of the body, solid or dispersed tissue types, etc.), especially when such cells or human cell containing compositions are to be transplanted into a human being. This includes, but is not limited to human organs or tissues or parts thereof from living or deceased human donors that are supported in a non-human mammal host. In one variation, the human organ(s) or tissue(s) is, at the time of transplantation to the non-human host, already functionally developed and may, for example, be from a donor at a post-birth stage of development. In a different variation, the human organ(s) or tissue(s) are, at the time of transplantation to the non-human host still be in an anlagen stage of development. Such anlagen may, upon transplantation to the host, continue their growth and differentiation into a functioning organ or tissue. Methods for transplanting anlagen to a host mammal for development are disclosed in U.S. Pub. Nos. 20040191228, 20040136972, 20040082064, 20030198628, 20030096016 and 20030086909, each of which is incorporated by reference herein in its entirety. Chimeric human, non-human mammal organs and tissues to be supported in the xeno-reduced animal host may be produced by any method, for example, by the facilitated cell replacement methods of Beschorner or by introduction of human cells into a non-human mammal, such as a fetus or a post-birth individual, without facilitated replacement, such as by the methods of U.S. Pub. Nos. 20020100065 (application Ser. No. 09/895,895) and 20030096410 (application Ser. No. (09/178,036).

Accordingly, a general method for culturing human cells in a non-human animal, such as a non-human mammal, is provided that includes the steps of: introducing human cells in any form (for example, a human organ or a human solid or dispersed tissue, or any human cell containing composition such as those described above) into a non-human host animal, such as a non-human mammal, that is at least substantially immunologically tolerant of the human cells and which is genetically modified, or otherwise modified or treated, to reduce the expression of at least one xenoantigen (defined with respect the human immune system), such as alpha-galactosyl epitopes and/or NeuGc epitopes, so that the amount of xenoantigens transferred to the human cells is reduced; and supporting the cells in the host animal for a period of time, such as at least 2 days, or at least one week or at least one month. A related method further includes the step of removing the human cells from animal host.

In the case that the host is modified to reduce the expression of alpha-galactosyl epitopes, it may for example be modified to reduce the expression of alpha (1,3) galactosyltranferase-synthesized and/or iGb3 synthase-synthesized alpha-galactosyl epitopes.

In another variation, a mixture of human cells and host cells is then removed from the host and the host cells are selectively killed to obtain an at least substantially pure composition of human cells. A host animal, or replacement cells, genetically modified to enable the selective deletion of the host cells as described herein may, for example, be used in this case.

In a different variation, the human cell containing composition is then removed from the animal host and is post-conditioned with at least one enzymatic treatment to remove host xenoantigens, as described herein above.

In still another variation, the human cell containing composition that was hosted in the non-human animal is removed from the host and placed on extracorporeal support, for example, for at least 1, 2, 3, 4, 7, or 30 days.

In a further variation of the embodiment or any of its aforementioned variations, the human cell containing composition, for example, a functionally developed human organ or tissue, can then be transplanted to a human patient in need thereof. In this manner, for example, human organs and tissues can be banked in a living state in non-human animal hosts until they are needed. In a related variation, the host is sized to support a functionally developed, solid human organ or tissue or at least a substantial portion thereof, such as a human liver, pancreas, kidney, heart or lung, with respect to a human child, adolescent or adult. For example, ungulate hosts, such as pigs, sheep and bovids, can be suitably sized.

Genetically modified animals are animals that include a genetic modification such as a mutation (e.g., a deletion, substitution, inversion, transposition and/or insertion) and/or a transgene (a transgenic animal) that was introduced in the current or in any prior generation. Modifications of a donor or host animal that reduce or completely eliminate the expression of a xenoantigen may be of any sort, such as genetic modifications or epigenetic modifications, and may be introduced in any current or prior generation so long as the host comprises the modification(s). Genetic modifications that inactivate a gene or an allele of a gene may be of any sort and may, for example, include mutations of the promoter of a gene that reduce or eliminate transcription of the gene and/or mutations in the normally transcribed sequence of gene that prevent expression of the transcript (such as elimination of a necessary start codon or ribosome-binding sequence) or prevent expression of a functional protein product. Genetic mutations of a gene sequence may be of any sort, such as deletions, insertions, substitutions, inversions and/or combinations thereof. Another kind of genetic modification of a host that can reduce the expression of a xenoantigen involves integration of a transgene into the host (in any current or prior generation), wherein the transgene produces a gene product, such as an RNA or protein, that has the effect of reducing the expression of the xenoantigen. In one example, a genomically integrated transgene that drives the expression (e.g., constitutive or inducible) of an RNA-silencing molecule that silences the mRNA transcripts of a selected gene, such as a gene for a protein xenoantigen or for a xenoantigen-producing enzyme, is used. In another example, a genomically integrated transgene that drives the expression (e.g., constitutive or inducible) of a gene that produces a gene product that cleaves or alters a xenoantigen is used. In still another example, a genomically integrated transgene that drives the expression (e.g., constitutive or inducible) of a gene that produces an enzyme that competes for substrate with a xenoantigen-producing enzyme can be used. Epigenetic modifications can also reduce or completely eliminate the activity of a gene. For example, double-stranded RNA-mediated gene silencing of a promoter of a gene and/or the other parts of the gene can silence transcription of the gene. While not being limited by theory, RNA-mediated gene silencing is believed to be mediated by methylation and/or other modifications of a gene at the DNA level.

The term human cells as referred to herein includes human cells and human-cell-derived cells. Examples include, but are not limited to, primary or cell culture passaged human cells, non-immortalized, immortalized or conditionally immortalized human cells, at least substantially human cells, genetically modified human cells, epigenetically modified human cells and unmodified human cells. In one variation of embodiments of the invention in which organs or tissues cellularly reconstituted with human cells are to be transplanted to a designated human recipient, the replacement cells used are derived from the designated recipient. In another variation of embodiments of the invention in which organs or tissues cellularly reconstituted with human cells are to be transplanted to a human recipient, human replacement cells having a predetermined HLA-type are used to cellularly reconstitute the organ or tissue so that the reconstituted organ or tissue can be matched to a recipient. In a subvariation, the HLA-type of an intended human recipient is determined and replacement cells at least substantially matching the HLA-type of the intended recipient are then used to cellularly reconstitute an organ or tissue according to the invention. In still another variation of embodiments of the invention in which organs or tissues cellularly reconstituted with human cells are to be transplanted to a human recipient, human replacement cells modified to lack one or more HLA determinants are used. In a subvariation, the human replacement cells are at least substantially universally acceptable with respect to HLA determinant barriers to transplantation.

Those skilled in the art will also appreciate that non-human mammals or cell lines that are used as hosts, donors and/or replacement cells or providers thereof according the invention can be genetically modified to eliminate any endogenous retroviruses that may be characteristically present in the genome of the animal or cell line. For example, swine lacking porcine endogenous retrovirus (PERV) may be used as mammal hosts, donors and/or replacement cell providers according to the invention.

Issued United States patents are identified herein with the prefix “U.S.” followed by the patent number. Published United States Patent Applications are identified herein with the prefix “U.S. Pub. No.” followed by the publication number. Each of the patents, patent applications, genetic sequences, articles and other publications cited in this disclosure is incorporated by reference in its entirety as if each was set forth herein.

The following U.S. patents, which may or may not be cited elsewhere in this disclosure, are incorporated by reference herein in their entireties: U.S. Pat. No. 6,923,959; U.S. Pat. No. 6,916,654; U.S. Pat. No. 6,911,220; U.S. Pat. No. 6,825,395; U.S. Pat. No. 6,794,182; U.S. Pat. No. 6,758,865; U.S. Pat. No. 6,734,295; U.S. Pat. No. 6,718,986; U.S. Pat. No. 6,700,037; U.S. Pat. No. 6,699,231; U.S. Pat. No. 6,689,598; U.S. Pat. No. 6,680,305; U.S. Pat. No. 6,677,150; U.S. Pat. No. 6,673,987; U.S. Pat. No. 6,673,594; U.S. Pat. No. 6,660,905; U.S. Pat. No. 6,558,663; U.S. Pat. No. 6,498,285; U.S. Pat. No. 6,649,595; U.S. Pat. No. 6,642,045; U.S. Pat. No. 6,639,122; U.S. Pat. No. 6,638,747; U.S. Pat. No. 6,627,393; U.S. Pat. No. 6,589,223; U.S. Pat. No. 6,582,953; U.S. Pat. No. 6,576,464; U.S. Pat. No. 6,562,619; U.S. Pat. No. 6,524,785; U.S. Pat. No. 6,521,448; U.S. Pat. No. 6,514,752; U.S. Pat. No. 6,506,549; U.S. Pat. No. 6,500,929; U.S. Pat. No. 6,495,735; U.S. Pat. No. 6,492,103; U.S. Pat. No. 6,491,912; U.S. Pat. No. 6,469,229; U.S. Pat. No. 6,447,767; U.S. Pat. No. 6,423,316; U.S. Pat. No. 6,368,572; U.S. Pat. No. 6,413,769; U.S. Pat. No. 6,368,572; U.S. Pat. No. 6,353,150; U.S. Pat. No. 6,342,344; U.S. Pat. No. 6,331,658; U.S. Pat. No. 6,331,319; U.S. Pat. No. 6,325,999; U.S. Pat. No. 6,296,846; U.S. Pat. No. 6,280,718; U.S. Pat. No. 6,258,998; U.S. Pat. No. 6,255,474; U.S. Pat. No. 6,252,136; U.S. Pat. No. 6,245,566; U.S. Pat. No. 6,215,039; U.S. Pat. No. 6,194,635; U.S. Pat. No. 6,187,757; U.S. Pat. No. 6,172,190; U.S. Pat. No. 6,166,288; U.S. Pat. No. 6,136,954; U.S. Pat. No. 6,132,708; U.S. Pat. No. 6,129,911; U.S. Pat. No. 6,100,443; U.S. Pat. No. 6,100,082; U.S. Pat. No. 6,093,872; U.S. Pat. No. 6,090,622; U.S. Pat. No. 6,060,049; U.S. Pat. No. 6,046,379; U.S. Pat. No. 6,046,046; U.S. Pat. No. 6,030,833; U.S. Pat. No. 6,018,096; U.S. Pat. No. 5,976,524; U.S. Pat. No. 5,965,433; U.S. Pat. No. 5,925,802; U.S. Pat. No. 5,912,411; U.S. Pat. No. 5,871,997; U.S. Pat. No. 5,866,757; U.S. Pat. No. 5,849,991; U.S. Pat. No. 5,821,117; U.S. Pat. No. 5,807,737; U.S. Pat. No. 5,752,929; U.S. Pat. No. 5,709,843; U.S. Pat. No. 5,672,346; U.S. Pat. No. 5,652,373; U.S. Pat. No. 5,643,746; U.S. Pat. No. 5,639,939; U.S. Pat. No. 5,633,426; U.S. Pat. No. 5,625,127; U.S. Pat. No. 5,599,659; U.S. Pat. No. 5,589,362; U.S. Pat. No. 5,586,438; U.S. Pat. No. 5,556,954; U.S. Pat. No. 5,523,226; U.S. Pat. No. 5,498,427; U.S. Pat. No. 5,494,822; U.S. Pat. No. 5,476,997; U.S. Pat. No. 5,418,147; U.S. Pat. No. 5,416,260; U.S. Pat. No. 5,362,622; U.S. Pat. No. 5,326,706; U.S. Pat. No. 4,837,390; U.S. Pat. No. 4,745,759; and U.S. Pat. No. 4,186,565.

The following published U.S. patent applications, which may or may not be cited elsewhere in this disclosure, are incorporated by reference herein in their entireties: U.S. Pub. Nos. 20050268347 (application Ser. No. 10/857,613); 20050266561 (application Ser. No. 10/996,217); 20050265995 (application Ser. No. 11/116,939); 20050223418 (application Ser. No 10/863,116); 20050201990 (Ser. No. 11/076,668); 20050177883 (Ser. No. 10/470,785); 20050176139 (Ser. No. 11/032,153); 20050170452 (Ser. No. 10/500,240); U.S. Pub. Nos. 20050164210(Ser. No. 10/763,479); 20050155095 (Ser. No. 10/981,935); 20050155094 (Ser. No. 10/503,464); 20050148072 (Ser. No. 10/944,919); 20050142121 (Ser. No. 10/949,411); 20050125853 (Ser. No.10/505,760); 20050120400 (Ser. No.10/499,407); 20050112122 (Ser. No. 10/933,933); 20050108783 (Ser. No. 10/947,920); 20050108780 (application Ser. No. 10/894,194); 20050076399 (Ser. No. 10/500,748); 20050028230 (Ser. No. 10/843,038); 20040268424 (Ser. No. 10/646,970); 20040258669 (Ser. No. 10/701,789); 20040209357 (Ser. No. 10/769,686); 20040191228 (Ser. No. 10/487,944); 20040180041 (Ser. No 10/809,556); 20040171824 (Ser. No. 10/469,881); 20040171155 (Ser. No. 10/762,888); 20040171138 (Ser. No. 10/640,867); 20040136972 (Ser. No. 10/759,033); 20040110286 (Ser. No.10/313,195); 20040073963 (Ser. No. 10/362,429); 20030224350 (Ser. No.10/113,664); 20030211098 (Ser. No. 10/181,896); 20030206891 (Ser. No. 10/341,967); 20030203427 (Ser. No. 10/125,994); 20030165480 (Ser. No. 10/135,919); 20030147859(Ser. No. 09/881,721); 20030131365 (Ser. No. 10/172,459); 20030115616 (Ser. No. 10/243,087); 20030096410 (Ser. No.09/178036); 20030092174 (Ser. No. 10/147,286); 20030086909 (Ser. No. 09/222,460); 20030068818 (Ser. No. 10/105,963); 20030068818 (Ser. No. 10/105,963); 20030049235 (Ser. No. 09/477,737); 20030014770 (Ser. No. 10/098,276); 20030068308 (Ser. No. 10/132,443); 20030003574 (Ser. No. 10/099,539); 20030003083 (Ser. No. 10/169,028); 20030198628 (Ser. No. 10/395,552); 20020197240 (Ser. No.10/146,092); 20020187972 (Ser. No. 09/949,278); 20020164571 (Ser. No. 09/798,790); 20020100065 (Ser. No. 09/895,895); 20020090370 (Ser. No. 09/753,007); 20020031494 (Ser. No. 10/254,077); 20010053362 (Ser. No. 09/802,350); and 20010049139 (Ser. No. 08/816,750).

In addition, the following published international applications and their related U.S. applications are each incorporated by reference in their entireties: International Pub. No. WO 2004/108904 A2 of PCT/US2004/018106 and U.S. Prov. Appin. Ser. No. 60/476,396 to which priority is claimed; International Pub. No. WO 2004/027029 A2 of PCT/US2003/029251, U.S. Prov. Appin. Ser. No. 60/411,790 to which priority is claimed, and the U.S. national phase Ser. No. 10/527,587; and International Pub. No. WO 2004/016742 A2 of PCT/US2003/025199 and U.S. Prov. Appin. Ser. No. 60/403,405 to which priority is claimed.

It should be understood that the embodiments and examples set forth within this disclosure are meant to illustrate various aspects of the invention and are not limiting of its scope. Many embodiments and variations within the spirit and scope of the invention may be apparent to those of skill in the art upon reviewing this disclosure.

What is claimed is:

1. A method for reconstituting a donor organ or tissue with replacement cells, comprising the steps of:
transplanting a solid organ or solid tissue or solid part thereof from a human or non-human mammal donor to a non-human mammal host that supports the donor organ or tissue or part thereof in a living state, wherein at least some of the cells of the donor organ or tissue are at least substantially selectively killable versus the host cells and the replacement cells;
selectively killing at least some of the endogenous donor cells of donor organ or tissue or part thereof after the donor organ or tissue or part thereof is transplanted to the host; and
introducing replacement cells into the donor organ or tissue or part thereof to replace endogenous donor cells of the transplanted organ or tissue or part thereof,
wherein, the step of selectively killing at least some of the endogenous donor cells promotes replacement of the donor cells by the replacement cells.

2. The method of claim 1, wherein:
the donor organ or tissue comprises a negative selection marker gene under control of a broad-activity promoter thereby rendering at least a substantial proportion of the cell types of the donor organ or tissue selectively killable in response to a set of one or more conditions;
the cells of the non-human host mammal and the replacement cells are not substantially killable in response to the set of one or more conditions that kills the donor cells; and
the step of selectively killing at least some of the donor cells comprises application of the set of one or more conditions.

3. The method of claim 2, wherein the negative selection marker gene is a suicide gene.

4. The method of claim 1, wherein the step of introducing replacement cells into the donor organ or tissue comprises introducing the replacement cells before transplanting the donor organ or tissue or part thereof into the host.

5. The method of claim 4, wherein the step of introducing replacement cells into the donor organ or tissue or part thereof comprises introducing the replacement cells into the donor organ or tissue before the donor organ or tissue or part thereof is removed from the donor mammal.

6. The method of claim 4, wherein the donor is a non-human mammal.

7. The method of claim 6, wherein the step of introducing replacement cells into the donor organ or tissue comprises introducing stem cells into the donor mammal during a fetal or neo-natal stage of development so that the solid organ or solid tissue or solid part thereof comprises replacement cells already incorporated therein before it is removed from the donor for transplantation to the non-human host mammal.

8. The method of claim 1, wherein the step of introducing replacement cells into the donor organ or tissue comprises introducing the replacement cells after transplanting the donor organ or tissue or part thereof to the host.

9. The method of claim 8, wherein the donor animal is a non-human mammal.

10. The method of claim 1, wherein the replacement cells comprise human cells.

11. The method of claim 10, wherein the replacement cells consist essentially of human cells.

12. The method of claim 1, wherein the organ or tissue is selected from the group consisting of: kidney, lung, heart, liver, and pancreas.

13. The method of claim 12, wherein the replacement cells comprise human cells.

14. A method for reconstituting a donor organ or tissue with replacement cells, comprising the steps of:
transplanting a solid organ or solid tissue or solid part thereof from a human or non-human mammal donor to a non-human mammal host that supports the donor organ or tissue or part thereof in a living state, wherein the growth of at least some of the cells of the donor organ or tissue is selectively impairable in response to a set of one or more conditions, and wherein the growth of endogenous cells of host cells is not substantially impairable by the set of one or more conditions;
selectively impairing the growth of at least some of the donor organ or tissue cells after the organ or tissue is transplanted into the host by applying the set of one or more conditions; and
introducing human replacement cells into the donor organ or tissue, wherein the growth of the replacement cells is not substantially impairable by the set of one or more conditions.

15. The method of claim 14, wherein the step of introducing human replacement cells into the donor organ or tissue comprises introducing the human replacement cells before the organ or tissue or part thereof is transplanted into the host.

16. A method for providing a non-human animal wherein a trait is limited to a preselected organ or tissue or part thereof supported by the animal, comprising the step of:
transplanting a preselected solid organ or solid tissue or solid part thereof from a human donor or a non-human animal donor to a non-human animal host, wherein the solid organ or solid tissue or solid part thereof is supported in a living state,
wherein at least some of the endogenous cell types of the transplanted donor solid organ or solid tissue or solid part thereof have a desired trait, and
wherein the endogenous cells of the host animal at least substantially do not have the trait.

17. The method of claim 16, wherein the trait comprises inducible or constitutive or developmentally regulated expression of a negative selection marker.

18. The method of claim 16, wherein the trait comprises susceptibility to cell-death or growth-impairment in response to a set of one or more known conditions.

19. The method of claim 16, wherein the trait comprises inducible or constitutive or developmentally regulated expression of a preselected transgene.

20. The method of claim 19, wherein the expression of the transgene is under the control of a broad-activity promoter.

21. The method of claim 19, wherein the preselected transgene is a preselected suicide gene or a preselected growth-impairing gene.

22. The method of claim 16, wherein the donor and host are non-human animals of the same species.

23. The method of claim 22, wherein the donor and host are non-human mammals of the same species.
